(19) United States
(12) Patent Application Publication
(10) Pub. No.: US 2025/0218554 A1
(43) Pub. Date: Jul. 3, 2025

(54) MACHINE LEARNING-DRIVEN FRAMEWORK FOR PREDICTING IONIC CONDUCTIVITY OF SOLID-STATE ELECTROLYTES

(71) Applicant: Quantum Generative Materials LLC, Austin, TX (US)

(72) Inventors: Vaidish Sumaria, San Jose, CA (US); Jacob Vikoren, Pacific Beach, WA (US); David Sommer, Duvall, WA (US); Takat Rawal, Austin, TX (US); Deeptanshu Prasad, Vancouver (CA)

(21) Appl. No.: 18/597,709

(22) Filed: Mar. 6, 2024

Related U.S. Application Data
(60) Provisional application No. 63/549,342, filed on Feb. 2, 2024.
(60) Provisional application No. 63/615,719, filed on Dec. 28, 2023.

(51) Int. Cl. G16C 20/70; G16C 20/20

(52) U.S. Cl. CPC G16C 20/70; G16C 20/20

(57) ABSTRACT

A system and method are provided for a machine-learning drive framework for predicting ionic conductivity of solid-state electrolytes. In use, the method and/or system may include receiving, at a machine learning system, two or more molecular structures from at least one structural dataset, where the two or more molecular structures relate to ionic mobility. Additionally, atomic weights are calculated for the two or more molecular structures, and the machine learning system is trained based on the two or more molecular structures, where the training relies on at least one intrinsic atomic feature and the calculated atomic weights for the two or more molecular structures. Further, a bias-correction is applied for the two or more molecular structures to improve the training of the machine learning system. Further, one or more molecular dynamics (MD) simulations are outputted, using the machine learning system, for the two or more molecular structures.

### CROSS-REFERENCE TO RELATED APPLICATIONS

This patent application claims the benefit of priority to: U.S. Provisional Patent Application No. 63/615,719 (GENMP002+/GENMP0003P1), entitled “MACHINE LEARNING-DRIVEN FRAMEWORK FOR PREDICTING IONIC CONDUCTIVITY OF SOLID-STATE ELECTROLYTES” filed Dec. 28, 2023, and U.S. Provisional Patent Application No. 63/549,342 (GENMP0005+/GENMP0005P1), entitled “UNDERSTANDING PHOTOCATALYTIC REDUCTION” filed Feb. 2, 2024, all of which are assigned to the assignee hereof; the disclosure of all prior applications are considered part of and are incorporated by reference in this patent application.

### FIELD OF THE INVENTION

The present invention relates to developing more efficient solid-state electrolyte battery components, and more particularly to developing models that can predict characteristics of one or more candidate electrolyte composites.

### BACKGROUND

Ionic conductivity of solid-state battery electrolytes plays an important role in battery performance, safety, longevity, efficiency etc.

Traditional molecular dynamics simulation models have limited accuracy compared to machine learning (ML) alternatives. The traditional solutions rely on predefined equations and parameters to describe the interactions between atoms and molecules, and have limitations when it comes to accurately modeling certain chemical phenomena, such as dispersion forces, hydrogen bonding, and highly flexible molecules. Such limitations can lead to inaccuracies in predicting properties and structures. Additionally, developing accurate empirical force fields often requires extensive experimental data for parameterization, which can be a labor-intensive and time-consuming process.

As such, there is thus a need to address these and/or other issues associated with the prior art.

### SUMMARY

In some aspects, the techniques described herein relate to a method including: receiving, at a machine learning system, two or more molecular structures from at least one structural dataset, wherein the two or more molecular structures relate to ionic mobility; calculating, using the machine learning system, atomic weights for the two or more molecular structures; training the machine learning system based on the two or more molecular structures, wherein the training relies on at least one intrinsic atomic feature and the calculated atomic weights for the two or more molecular structures; applying, using the machine learning system, a bias-correction for the two or more molecular structures; and outputting, using the machine learning system, one or more molecular dynamics (MD) simulations for the two or more molecular structures, wherein the one or more MD simulations including validating associated structural energy or atomic forces for the two or more molecular structures.

In some aspects, the techniques described herein relate to a method, wherein the two or more molecular structures include at least one of: atomic positions, associated structural energy, or atomic forces affecting one or more atomic structures of the two or more molecular structures.

In some aspects, the techniques described herein relate to a method, wherein the receiving includes initial data obtained using ab-initio simulation via a density functional theory (DFT) calculation.

In some aspects, the techniques described herein relate to a method, wherein the training includes converting the two or more molecular structures into one or more mathematical descriptors.

In some aspects, the techniques described herein relate to a method, wherein the converting includes using translationally invariant functions.

In some aspects, the techniques described herein relate to a method, wherein the converting includes using rotationally invariant functions.

In some aspects, the techniques described herein relate to a method, wherein the training includes using a machine learned interatomic potentials (MLIP) model using a traditional ensemble.

In some aspects, the techniques described herein relate to a method, wherein the training includes using a machine learned interatomic potentials (MLIP) model using a diverse ensemble.

In some aspects, the techniques described herein relate to a method, wherein the at least one factor includes at least one of: atomic positions, associated structural energy, or atomic forces affecting the two or more molecular structures.

In some aspects, the techniques described herein relate to a method, wherein the outputting includes evaluating data from the training with an ensemble of networks to evaluate uncertainty levels against known threshold values.

In some aspects, the techniques described herein relate to a method, wherein the threshold is a known eV/angstrom value for atomic forces.

In some aspects, the techniques described herein relate to a method, wherein the threshold is a known me V/angstrom value for structural energy.

In some aspects, the techniques described herein relate to a method, wherein the evaluating of uncertainty levels against known threshold values indicates a level of uncertainty in ionic conductivity of the ionic mobility.

In some aspects, the techniques described herein relate to a method, further comprising adding one or more new structural datasets to a first database.

In some aspects, the techniques described herein relate to a method, further comprising adding the one or more new structural datasets to a second database that is different from the first database.

In some aspects, the techniques described herein relate to a method, wherein the one or more new structural datasets in the second database are re-evaluated using a DFT process and are outputted as additional molecular structures in the at least one structural dataset.

In some aspects, the techniques described herein relate to a method, wherein the MD simulations for the two or more molecular structures includes increasing a temperature of the one or more MD simulations.

In some aspects, the techniques described herein relate to a method, wherein the MD simulations for the two or more molecular structures includes decreasing a temperature of the one or more MD simulations.

In some aspects, the techniques described herein relate to a method, wherein the MD simulations for the two or more molecular structures includes determining an activation energy based on increasing or decreasing a temperature of the one or more atomic structures.

In some aspects, the techniques described herein relate to a method, at least one of the validated associated structural energy or the validated atomic forces is used to improve a model associated with the machine learning system, wherein the model is machine learned interatomic potentials (MLIP).

### BRIEF DESCRIPTION OF THE DRAWINGS

FIG. 1A shows an illustration of ionic mobility, in accordance with one embodiment.

FIG. 1B illustrates a system for predicting ionic conductivity of solid-state electrolytes, in accordance with one embodiment.

FIG. 2 illustrates a method for predicting ionic conductivity of solid-state electrolytes, in accordance with one embodiment.

FIG. 3 illustrates a system for automated reiterative machine learned interatomic potentials (MLIP) training, in accordance with one embodiment.

FIG. 4 illustrates a system for utilizing MLIP with accelerated molecular dynamics (MD) simulation to calculate the ionic conductivity and activation energy of conductive ion transport in an atomic structure, in accordance with one embodiment.

FIG. 5 illustrates a system for developing molecular structure training data, in accordance with one embodiment.

FIG. 6 illustrates a collection of possible industrial applications of solid-state electrolyte ionic conductivity maximization, in accordance with one embodiment.

FIG. 7A illustrates an environment in which various functions of the present disclosure may operate, in accordance with one embodiment.

FIG. 7B illustrates a dataset creation/loading and machine learning model training environment, in accordance with one embodiment.

FIG. 7C illustrates a prediction subsystem environment, in accordance with one embodiment.

FIG. 7D illustrates a generative training model subsystem environment, in accordance with one embodiment.

FIG. 7E illustrates a material selection and validation subsystem environment, in accordance with one embodiment.

FIG. 7F illustrates atomic structure conditions and environments, in accordance with one embodiment.

FIG. 7G illustrates a multi-layer perceptron environment, in accordance with one embodiment.

FIG. 7H illustrates a hardware and software architecture for a generative atomistic design (GAD) application, in accordance with one embodiment.

FIG. 7I illustrates a relationship between atomic structure training and inference data, in accordance with one embodiment.

FIG. 7J illustrates user and expert interactions with computing platforms, in accordance with one embodiment.

FIG. 8 illustrates a network architecture, in accordance with one possible embodiment.

FIG. 9 illustrates an exemplary system, in accordance with one embodiment.

### DETAILED DESCRIPTION

Currently, generating novel materials that maximize ionic conductivity involves extremely time-consuming efforts that often involve running a number of experiments that are designed based on expert knowledge, and then refining those experiments and adjusting those experimental parameters in order to approach a more optimal solution and outcome. The outcome in question is a physical material that retains the properties of interest, but which must undergo many cycles of experimentation and evaluation to determine the resulting material's level of compliance and utility. As such, previous efforts to perform advanced materials research and development are typically slow and lead to protracted development cycles. Such efforts inherently bring with them greater data generation demands and higher computational costs and are limited in the complexity and scale of systems that they can address.

In view of such considerations, the present disclosure introduces a generative design workflow for producing electrolyte battery components that maximize ionic conductivity. The workflow trains a machine learning model (such as machine learned interatomic potentials MLIP) via automated ensemble-driven testing and evaluation. Such workflow may be used to accelerate the research and optimization of known materials, as well as foster the generative discovery of novel functional materials by taking advantage of GPU acceleration to assist simulations for ionic conductivity. By leveraging density function theory (DFT) models and publicly available data, iterative training algorithms may predict novel materials that optimize ionic conductivity. Furthermore, the system is able to leverage “learning on the fly” active learning cycles (by deciding when to retrain the model during molecular dynamics simulations) to enhance model accuracy and reduce the data requirements for training these models. Such a workflow may decrease data generation requirements and may extend molecular dynamics simulation capacity to accommodate larger systems and enables generative design of electrolyte battery components that maximize ionic conductivity.

Further, it is noted that traditional molecular dynamics simulation models have limited accuracy compared to machine learning (ML) alternatives. ML alternatives, on the other hand, use many orders of magnitude more parameters than traditional force field models, and tend to be more accurate.

Because of the limited availability of comprehensive interatomic potentials across a broad chemical spectrum, ab-initio molecular dynamics (AIMD) may be used for the exploration of new ionic conductors. Due to computational demands, however, AIMD simulations may be typically constrained to time scales ranging from only tens to hundreds of picoseconds. As the speed of cation migration decreases exponentially with higher migration energies, AIMD may be suitable for determining conductivity at room temperature when migration energy is below approximately 0.2 eV. However, even for materials with such low migration energies, there can be notable errors in predicting diffusivity. As such, simulations, as disclosed herein, can be conducted at elevated temperatures to model materials with higher migration energies, and such simulations can be employed for extrapolating room temperature conductivity if no phase transformations occur between room temperature and the temperatures at which AIMD simulations are conducted.

Currently, ab-initio molecular dynamics (AIMD) simulations may provide a comprehensive view of ionic motion that may offer insights into diffusion properties (such as ionic conductivity, activation energy, and thermal effects) beyond migration barriers in a given structure. It is recognized that AIMD may have limitations due to high computational cost, compute time, grain-boundary component, less conductive phases in the sample, etc. However, AIMD, as used within the present disclosure, may be used in a way to overcome such deficiencies.

It should also be noted that existing machine learned interatomic potentials (MLIP) models suffer from highly inhomogeneous feature-space sampling in the training set. As a result, underrepresented atomic configurations, often critical for simulations, cause large errors even though they are included in the training set. To address these issues, the disclosure herein involves developing a machine learning model (such as MLIP) that corrects these inherent training biases and is capable of running faster molecular dynamics (MD) simulations of larger systems with grain boundaries to get accurate and fast estimations of ionic conductivity. Machine learning (ML) driven MD simulations also clarify the mechanism of ionic transport, which can further assist in developing newer materials with better ionic conductivity.

As such, the disclosure here rectifies known issues and provides a way for more accurately modeling and predicting ionic conductivity.

### Definitions and Use of Figures

Some of the terms used in this description are defined below for easy reference. The presented terms and their respective definitions are not rigidly restricted to these definitions—a term may be further defined by the term's use within this disclosure. The term “exemplary” is used herein to mean serving as an example, instance, or illustration. Any aspect or design described herein as “exemplary” is not necessarily to be construed as preferred or advantageous over other aspects or designs. Rather, use of the word exemplary is intended to present concepts in a concrete fashion. As used in this application and the appended claims, the term “or” is intended to mean an inclusive “or” rather than an exclusive “or”. That is, unless specified otherwise, or is clear from the context, “X employs A or B” is intended to mean any of the natural inclusive permutations. That is, if X employs A, X employs B, or X employs both A and B, then “X employs A or B” is satisfied under any of the foregoing instances. As used herein, at least one of A or B means at least one of A, or at least one of B, or at least one of both A and B. In other words, this phrase is disjunctive. The articles “a” and “an” as used in this application and the appended claims should generally be construed to mean “one or more” unless specified otherwise or is clear from the context to be directed to a singular form.

Various embodiments are described herein with reference to the figures. It should be noted that the figures are not necessarily drawn to scale, and that elements of similar structures or functions are sometimes represented by like reference characters throughout the figures. It should also be noted that the figures are only intended to facilitate the description of the disclosed embodiments-they are not representative of an exhaustive treatment of all possible embodiments, and they are not intended to impute any limitation as to the scope of the claims. In addition, an illustrated embodiment need not portray all aspects or advantages of usage in any particular environment.

An aspect or an advantage described in conjunction with a particular embodiment is not necessarily limited to that embodiment and can be practiced in any other embodiments even if not so illustrated. References throughout this specification to “some embodiments” or “other embodiments” refer to a particular feature, structure, material or characteristic described in connection with the embodiments as being included in at least one embodiment. Thus, the appearance of the phrases “in some embodiments” or “in other embodiments” in various places throughout this specification are not necessarily referring to the same embodiment or embodiments. The disclosed embodiments are not intended to be limiting of the claims.

### Descriptions of Exemplary Embodiments

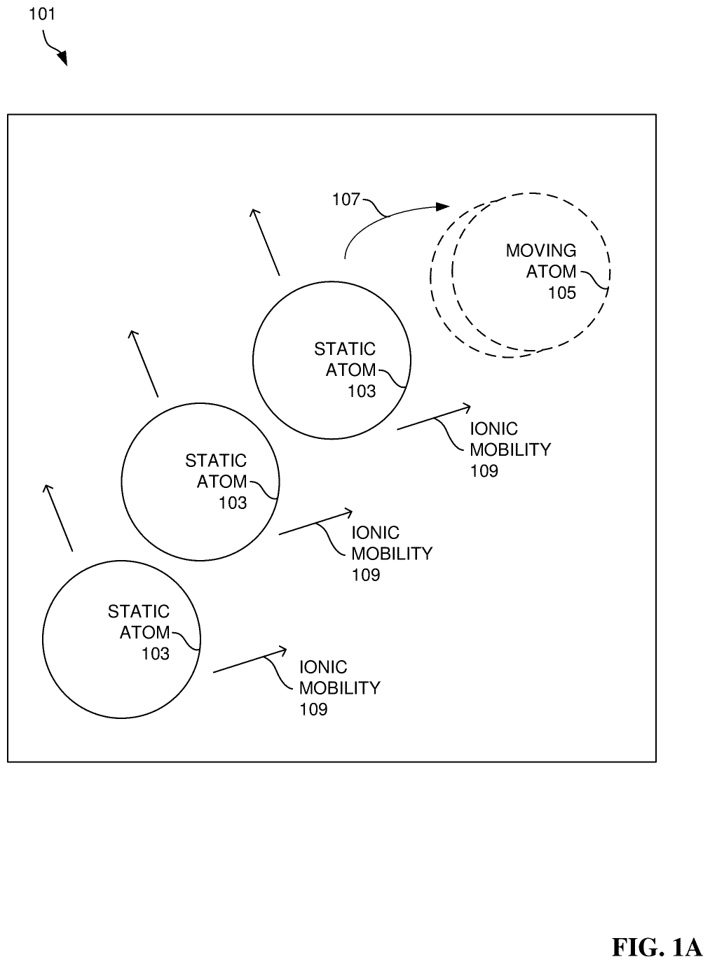

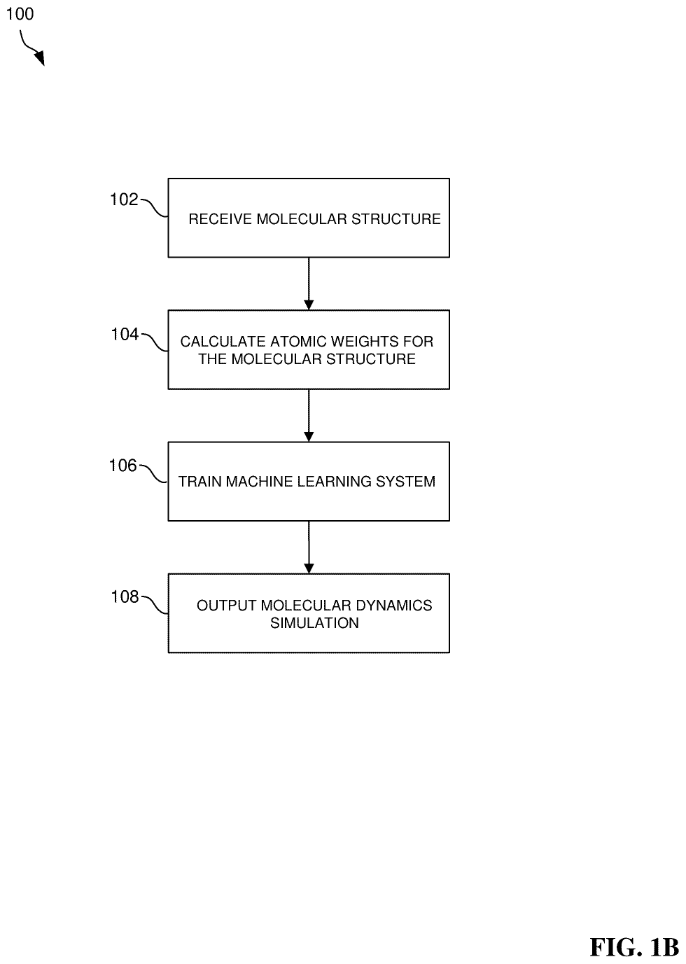

FIG. 1A shows an illustration 101 of ionic mobility, in accordance with one embodiment. It is to be understood that ionic mobility may be synonymous with ionic conductivity, and may relate to atomic movement. Additionally, an atomic weight of a molecule may be directly related to the ionic mobility, as the atomic weight may affect the ability of ions to move (due to size and/or density of ions and the interactions of ions with the surrounding environment).

As shown, the illustration shows multiple static atoms 103, a moving atom 105, a movement 107, and ionic mobility 109. In particular, the ionic mobility 109 may be determined particularly for the moving atom 105 due to the movement 107.

In seeking to track ionic mobility, it is recognized that conventional systems have great limitations. For example, if the illustration 101 represented billions of atoms, being able to compute and track simultaneously the movement of all atoms would overwhelm nearly any computing system. Thus, many systems may be configured to track a single atom (such as the moving atom 105).

Ionic conductivity measurements may occur by tracking a motion of moving atoms within an electrolyte. For example, in the case of a Li-ion battery, the atom to be tracked may be a lithium Li ion. Ionic conductivity therefore may be calculated by tracking of individual cation motion within an electrolyte structure.

It recognized that machine learning systems, and in greater detail, machine learned interatomic potentials (MLIP), may be used to assist in modeling and calculating ionic conductivity measurements. However, it is also recognized that a bias (associated with the ionic conductivity measurements) may be introduced with respect to training an MLIP based on ab-initio and/or DFT generated datasets.

As such, the present disclosure assists with removing such bias, and introducing methods and systems for modeling and calculating ionic conductivity measurements.

In particular, the present disclosure may include receiving input data that relates to two or more molecular structures. Such molecular structures may be correlated with ionic mobility. A machine learning system may be trained based on the two or more molecular structures. Further, atomic weights may be calculated for the two or more molecular structures, and one or more molecular dynamics (MD) simulations may be outputted for the two or more molecular structures. Such simulations may include validating associated structural energy or atomic forces for the two or more molecular structures.

In this manner, the computed atomic weights can be utilized in the training of the machine learning potential to reduce the inherent bias, allowing for greater accuracy and fidelity of the ML model. This furthermore ensures reliable molecular dynamic simulations that can help predict ionic conductivity accurately.

FIG. 1B illustrates a system 100 for predicting ionic conductivity of solid-state electrolytes, in accordance with one embodiment. As an option, the system 100 may be implemented in the context of any one or more of the embodiments set forth in any previous and/or subsequent figure(s) and/or description thereof. Of course, however, the system 100 may be implemented in the context of any desired environment. Further, the aforementioned definitions may equally apply to the description below.

As shown, two or more molecular structures are received from at least one structural dataset, where the two or more molecular structures relate to ionic mobility. See operation 102. It is to be noted that, within the context of the present description, details relating to the one or more molecular structure, and in particular the process used to generate molecular structure training data are addressed below with the detailed description of subsystem 500 described hereinbelow.

In one embodiment, the molecular structures that are received from the at least one dataset include, but not be limited to, atomic positions, associated structural energy, atomic weights, atomic forces affecting one or more atomic structures of the two or more molecular structures, and/or a combination thereof. In a more detailed embodiment, the molecular structure datasets may include three key aspects: the structure itself (e.g., the Cartesian coordinates coinciding with the location(s) of the atoms' positions), the energy associated with a given structure, and forces acting on the atoms of the structure. As such, the three elements may function as input parameters within the context of operation 102. It should be noted that, in so doing, the Cartesian coordinates of the candidate model may be converted to mathematical descriptors. For example, even where a given set of atoms' Cartesian coordinates may change, the overall system may essentially remain the same. Such a state may reflect the role the mathematical descriptors may play in the training system.

In another embodiment, the three elements (atomic positions, energy, and forces) may originate from density functional theory (DFT) calculations, and the atomic weights may then be calculated based on those structures' relative positions. Additionally, all four elements then become resources designed for training a machine learning model (such as the MLIP).

In still another embodiment, the molecular structures that are received from the at least one dataset may include initial data obtained using ab-initio (AIMD) simulation via the DFT process. For purposes of the present description, AIMD may be thought of as having two aspects: one may be the number of atoms being simulated and the other may be the time period during which the simulation is taking place. As such, an AIMD simulation may address the manner in which atoms interact with one another over an amount of time adequate to perform the necessary calculations. Further, AIMD simulation may be used to accurately predict a material's electronic structure, energetics, and various properties, which in turn may allow greater understanding regarding the intricacies of atomic and molecular interactions.

In a related embodiment, ionic conductivity may be predicted by running large timescale simulations. For example, movements of lithium ions within a Li+ battery may be observed over a time. In conventional AIMD (ab-initio molecular dynamics) systems, the time available to observe and analyze movement(s) may become limited. However, MD (molecular dynamics) within the context of a machine learning environment may allow for observation and calculations over a much longer period of time using, while essentially using the same amount of computing resources as previously employed when performing AIMD alone without a machine learning environment. It is further recognized that AIMD includes data which may result from DFT calculations (which can be time-consuming). As such, MD in combination with a machine learning environment, can output more results without significantly increasing (and in fact it may remain the same or decrease) power consumption.

In an alternative embodiment, AIMD may be thought of as a technique to obtain DFT datasets for training. In one embodiment, AIMD may take a snapshot of a molecular structure, run DFT calculations on that structure, and output three things, similar to what was discussed above: Cartesian coordinates, energies, and atomic forces for that particular snapshot. Next, AIMD may be used to assume a temperature and pressure acting on these systems and AIMD may move and/or modify the placement of the atoms around, which in turn may create a new configuration. This new configuration of the snapshot can in turn be evaluated using DFT to obtain the energy and forces for that new snapshot. In one embodiment, these criteria may then be processed with a set of multiple DFT calculations to result in training data (which may be subsequentially used by machine learning systems as indicated below).

In yet another embodiment, a framework for accurately measuring ionic conductivity of solid-state battery components may use one or more computational models that may minimize the number of experiments required. In a related embodiment, a framework may comprise machine learned interatomic potentials (MLIP). Traditional interatomic potentials, such as the Lennard-Jones potential or the Morse potential may be used. While these models can provide reasonably accurate results for many systems, their limitations increase as the systems become more complex. Conversely, an MLIP approach using machine learning techniques, particularly neural networks, and other regression algorithms, may be used to model the interactions between atoms in a material and overcome the limitations noted. In one embodiment, these interactions between atoms may be described by interatomic potentials, which include mathematical functions used to calculate the potential energy and atomic forces of a system of atoms as a function of their positions.

In the context of this disclosure, machine learned interatomic potentials (MLIP) may refer to any machine learning model that represents the interactions between atoms. For example, MLIP may include machine learning techniques that represent the potential energy surface of a system of atoms. In one embodiment, the potential energy surface may describe the energy of a system as a function of the atomic coordinates.

In legacy methods (e.g., DFT-based methods) simulation of atomic interactions may require complex and computationally intensive calculations. However, the MLIP methods disclosed herein may train machine learning models with one or more datasets of known atomic configurations and their corresponding/associated energies and forces. Once trained, the trained machine learning models may be used to rapidly predict the behavior of atomic systems under different conditions, making it vastly more efficient, computationally and timewise, compared to legacy methods. In the context of the present disclosure, MLIP methods may be used to enable simulation of larger systems or longer timescales than are feasible with legacy methods.

As shown, based on the molecular structure received, atomic weights may be calculated. See operation 104. In various embodiments, atomic weight may include a mass of a molecular structure. For example, the molecular structure may include a spatial arrangement of atoms. It is to be appreciated that the molecular structure may include any number of atoms, nucleus, protons, neutrons, and/or electrons. Further, molecular structure may include molecular geometry (involving bond angles and lengths), providing details on a 3D arrangement. For instance, water (H₂O) adopts a bent structure due to lone pairs on the central oxygen atom, while methane (CH₄) exhibits a tetrahedral shape with four equivalent C—H bonds.

Additionally, a machine learning system based on the two or more molecular structures is trained, where the training relies on at least one factor. See operation 106. In one embodiment, the at least one factor may include at least one of: atomic positions, associated structural energy, atomic weights, or atomic forces affecting the two or more molecular structures.

In another embodiment, the training process may include converting the two or more molecular structures into one or more mathematical descriptors. Additionally, such converting may include using translationally invariant functions and/or rotationally invariant functions. In the context of this present description, a rotationally invariant function may be observed where the value of the function does not change when arbitrary rotations are applied to one or more pertinent arguments. Further, it should be noted that both radial (constructed as sums of two-body terms) and angular (containing sums of three-body terms) symmetry functions may be employed, which may be comprised of input from different atoms in a molecular structure.

In one embodiment, where a particular molecular structure may be analyzed multiple times (for instance, using a first image and a third image of the same molecular environment), an increased density may be indicated with regard to observed atomic weights calculated by a Gaussian Density Function (GDF). That is, the GDF (shown below as Eq. 1) may observe the same image multiple times and, thus, record increased density. Additionally, the GDF may relate to training the machine learning system configured to simulate molecular dynamics in a material to compute ionic conductivity of the material. In particular, the GDF may be used to calculate atomic weights which may in turn be used to understand the molecular structural data. The GDF is:

$$ρ ( G ) = \frac{1}{M} {\sum }_{j = 1}^{M} exp ( - \frac{1}{2 {σ}^{2}} \frac{{\vert G - {G}_{j} \vert}^{2}}{D} ) \qquad Eq . 1$$

Where σ is the gaussian width, D is the dimension of the symmetry function vector (i.e., G∈R^{D}), M total number of atoms in the training data.

Using the gaussian density function (GDF), the atomic weight may be calculated, using Eq. 2, by passing the inverse of ρ(G) through a monotonically increasing function (modified sigmoid function chosen here).

$$Θ ( x ) = \frac{A x}{1 + {e}^{- b x + c}} x = \frac{1}{ρ ( {G}_{j} )} \qquad Eq . 2$$

Where A is a normalizing constant making the average of θ to be 1 and b, c are hyperparameters that are fine tunes for balanced training of the MLIP. Based on such calculation, the structural properties-positions, structural energy, atomic forces, and atomic weights—may be passed as training data to the machine learning model system (including MLIP). Within the context of the present description, intrinsic atomic feature may include the structural properties. Further, intrinsic atomic features may encompass essential characteristics inherent to individual atoms within chemical elements, defining their identity and behavior. These intrinsic atomic features may further include the atomic number, the atomic mass, electron configuration, electron energy levels, valence electrons, ionization energy, ion formation, electronegativity, atomic radius, etc., any and/or all of which may influence properties, reactions of the atom.

As a next step in the process, the bias may then be removed by introducing the inverse of the density-propagating condition. For example, a molecular environment that may have been observed 10 times would be weighed at only 1/10 of its value for the calculation algorithm. Therefore, it should be noted that training one or more molecular structure datasets may entail inversely focusing analytic resources on atomic structures. That is, where many cycles (or steps) of analysis note no changes in atomic structure, fewer resources, and thus proportionally less weight, should be given to areas of analysis that feature no changes. As such, any places where the analytic tools note changes in atomic structure should receive increased levels of scrutiny, and thus increased resource devotion. In another embodiment, monotonically increasing function, which is a modified function, may be employed, where the X on an organically increasing function is 1/X (one over the density just observed). For example, if an atomic environmental condition is observed 10 times, its actual weight may be 1/10 for mathematical purposes. Put another way, if the same thing is being observed again and again, it should be given decreased weight; if something is observed less frequently, it is thus axiomatically given greater weight. As such, therefore, the bias may be quantified using the Eq. 1 (Gaussian density function).

In another embodiment, the training process may include training a machine learned interatomic potentials (MLIP) model using a traditional and/or diverse ensemble. In one embodiment, for a model training process using machine learning, error estimation capability may be used and prioritized. As such, ensembles may help train on a given dataset multiple times and output multiple outputs for each. Additionally, ensemble methods may be thought of as processes that may create multiple potential predictive models, and combine those models to produce a more homogenous result. In known embodiments, ensemble methods, in a machine learning context, may be used to produce more accurate solutions than any single model might.

In one embodiment, a traditional ensemble may be used where certain parameters of a machine learning model are changed and the model may be trained (and re-trained) multiple times. In one example, five different networks may be trained on the same data, and different weights for each of these training output may be observed. During a validation test, a value may be tested across all of the datasets in the ensemble, and if the results are consistent across five or ten models that have been trained, it may be inferred that the system is not performing any extrapolation (i.e. the results are more predictable and favorable). Alternatively, if the result from the five or ten independently trained models are notably different, then it may be inferred that the system is very likely extrapolating and new model design is likely required. As such, an internal verification of the model design may ensure that the trained models are corrected trained.

Additionally, in one embodiment, a diverse ensemble (similar to a traditional ensemble) may be used. It is to be appreciated that the diverse ensemble may allow for testing in an alternative manner. For example, instead of testing against five completely different networks, two completely different networks may be involved and some of the hyper parameters of the system may be changed in the three other networks. Thus, the architecture of the machine learning model may be preserved, but may be modified and arranged as desired and/or consistent with the training data that is initialized (such as where three random values are given as initialization). As such, in this particular embodiment, the system may feature five networks, but two of the networks may have different architectures (as they are based on two different networks), while the remaining three may have the same architecture as one of the two diverse networks but initialized in a different manner.

In one embodiment, to ensure homogeneous and uniform training of the MLIP, the atomic weights calculated previously may be used. In addition, the training of atomic species with lower forces may be emphasized by weighing the force losses with the inverse of the magnitude of force on the atom, wherein a modified loss function equation is show in Eq. 3:

$$Γ = \frac{1}{N} {\sum }_{i = 1}^{N} {( \frac{{E}_{i}^{D F T} - {E}_{i}^{M L I P}}{{n}_{i}} )}^{2} + \frac{μ}{3 M} {\sum }_{j = 1}^{M} ( Θ [ \frac{1}{ρ ( {G}_{j} )} ] + \frac{1}{\vert {F}_{j}^{D F T} \vert} ) {\vert {F}_{j}^{D F T} - {F}_{j}^{MLIP} \vert}^{2} \qquad Eq . 3$$

Where Eᵢ^{DFT} and Eᵢ^{MLIP} denote the energy of structure i using the reference DFT calculation and that predicted by the MLIP. Fⱼ^{DFT} and Fⱼ^{MLIP} is the force on atom j similarly from the reference DFT calculation and that predicted by the MLIP. M is the total number of atoms in the training data and μ is the hyperparameter that defines the ratio of energy and force errors.

With respect to addressing biases arising from the redundancy of atomic environments in the training set, it is noted that non-uniformity in training on atomic forces may be found. The existing loss functions for the MLIP training may treat the absolute error in forces as constant, irrespective of force magnitude, leading to higher relative force errors for smaller values. To overcome the identified issues, Eq. 3 may be used in the MLIP model thereby ensuring improved and uniform training (such as for the ab-initio DFT data).

In a continued embodiment, once initial data is trained using an ensemble of networks, such network may be used to perform molecular dynamics (MD) simulations to generate new data. In such a process for example, in one embodiment, every fifth structure generated using the MD simulation may be evaluated with the ensemble of networks to evaluate the uncertainty in the predicted energy and forces. If the uncertainty in the measurement is higher than 0.2 eV/Å (angstrom) for forces or higher than 10 meV/angstrom for energy, that structure may be added to a second database and may be re-evaluated with ab-initio DFT calculations. In an embodiment, once 100 structures (or any predetermined amount) have been stored in the second database, the MD simulation may be stopped, and those structures in the second database may be re-evaluated using DFT and passed back to a structural database to be trained again in an iterative manner.

In a further embodiment, atomic-weighted training of the MLIP may result in a more robust and accurate model by reducing an inhomogeneous feature-space sampling in a training set. Thus, an atomic weight may be calculated where a position, energy, and force are known. Additionally, to calculate an atomic weight, per Eqs. 1 and 2, an atomic environment may be compared against other atoms within the training set. By way of practical example, if X number of structures have been trained, another number (Y) of structures may be applicable for further analysis and retraining, and a segment of those structures may go through DFT again. If that sample set of structures is observed to yield confirming results with regard to the existing model and screening algorithm, then those datasets may be put forth as suitable output data. Alternatively, if the sample set of structures is observed to render results outside of the known threshold parameters, then the one or more datasets may be reiteratively cycled back into the DFT process for further training until a convergence is measured using the overall model.

It is to be emphasized that the movement of the atoms in the molecular structure, and the accurate recording thereof, may be ranked as one the most important aspect of the training process. For example, the movement of the atoms may ultimately impact the ionic conductivity (similar to electrical elements that may move and change the molecular environment). As such, a greater amount of resources may be devoted to recording the movement of the atoms in the molecular structure during training. In an embodiment, where it is observed that one or more particular atoms may not be moving, the machine learning model may be set up such that those non-moving atoms are devoted fewer resources while the atoms in motion become a primary focus of observation and calculation. Thus, all the atomic weights and respective biases being observed with regard to the candidate molecular structure may be, axiomatically, more directed toward the moving atoms where there is a changing environment, rather than the constant, static atoms which may not affect the dynamics of the system. As such, the movement of the atoms may dictate the focus of data that is collected and used to train the machine learning system.

In the context of the present disclosure, the term bias may refer to a condition where the sum of elements of a single calculation focus remain static, and/or a series of calculations on a single aspect of the molecular structure that remains static. In other words, the machine learning model may observe a prevalent collection of atoms multiple times and may treat them as “important” to the calculations, whereas, it is noted that the non-static atoms or elements should be the basis of greater deference/attention by the generative atomistic design (GAD) application analysis process. For example, recorded data may be “biased” toward environments where analysis resources are focused on one or more areas of relative inactivity in a given structure, as opposed to the portion of the structure that demonstrates the associated properties the system is actually designed to seek and simulate. Thus, it is important that an “unbiased” approach is employed for machine learning model development to ensure an accurate predictive model may be produced.

Further, one or more molecular dynamics (MD) simulations are outputted. See operation 108. In one embodiment, the outputting of the simulations may include evaluating data from the training with an ensemble of networks to evaluate uncertainty levels against known threshold values. In one embodiment, the threshold may be a known eV/angstrom value for atomic forces and a known meV/angstrom value for structural energy, and the evaluating of uncertainty levels against known threshold values may be indicative of a level of uncertainty in ionic conductivity. As such, once initial data is trained using an ensemble of networks, as shown previously, such network may be used to perform molecular dynamics (MD) simulations to generate new data.

In another embodiment, the performing may include adding one or more new structural datasets to a first database. Additionally, the performing may include adding the one or more new structural datasets to a second database that is different from the first database. Additionally, the one or more new structural datasets in the second database may be re-evaluated using a DFT process and may be outputted as additional molecular structures in the at least one structural dataset. For example, every fifth structure generated using the MD simulation may be evaluated with the ensemble of networks to evaluate the uncertainty in the predicted energy and forces. If the uncertainty in the measurement is higher than 0.2 eV/Å for forces or higher than 10 meV/angstrom for energy, that structure may be added to a second database and may be re-evaluated with ab-initio DFT calculations. In an embodiment, once 100 structures (or any predetermined number) have been stored in the second database, the MD simulation may be stopped, and those structures in the second database may be re-evaluated using DFT and passed back to a structural database to be trained again in an iterative manner.

In practice, a trained MLIP may be used to perform one or more MD simulations using a Large-scale Atomic/Molecular Massively Parallel Simulator (LAMMPS) package. Such a process may use a 2 femtosecond time step where, for a given material, initial MD simulation may be performed at room temperature and ambient pressure for 1 nanosecond (or any predetermined amount of time). As such, the results of the simulation may then be used to calculate the mean squared displacement (MSD) of atoms, shown below in Eq. 4.

$$M S D ( t ) = \frac{1}{N} {\sum }_{i = 1}^{N} {\vert Δ {\vec{r}}_{i} ( t ) \vert}^{2} \qquad Eq . 4$$

where N is the number of mobile ions (Li) and Δ\vec{r}ᵢ(t)=\vec{r}ᵢ(t)−\vec{r}ᵢ(0)

In an embodiment, if the MSD is low (<3 Å²), i.e., the ionic species may not be mobile enough at room temperature, the temperature may be increased by 100K and MD simulation may be restarted until MSD>3 Å². If the temperature reaches 800K and the MSD remains below 3 Å², the candidate material may not be conductive enough at room temperature and the process may be terminated. It should be noted that this circumstance may serve as a first filter to rule out unlikely room temperature conductors (including Li+ conductors).

In a further embodiment, once MDS is above 3 Å², MD simulation may be run for a longer time (such as 4 ns and/or any predetermined length of time) and the Li ion MSD is measured. If at the high temperature, the MDS is lower than 50 (and/or any predetermined level), the candidate material is disregarded. If the MSD>50 (and/or any predetermined level), diffusivity shown as Eq. 5 and ionic conductivity shown as Eq. 6 (Nernst-Einstein equation) may calculated at that temperature:

$$D = {lim}_{t \to ∞} \frac{1}{2 dt} 〈 M D S ( t ) 〉 \qquad Eq . 5$$

$$σ = \frac{N {q}^{2}}{V {k}_{B} T} D \qquad Eq . 6$$

where d=3 is dimensions, MDS(t) indicates the ensemble average, N is number of Li atoms, V is the volume, q is the ion electric charge, and Tis the temperature.

In a likewise embodiment, the activation energy may be determined for the ionic conduction, the process for which may be repeated at lower temperatures (until room temperature) using an Arrhenius equation (Eq. 7):

$$D = {D}_{0} {e}^{- {E}_{a} / {k}_{B} T} \qquad Eq . 7$$

It is to be appreciated that Eq. 5 and Eq. 6 may be used to understand more fully the mobility of ions. Further, Eq. 7 may be used to understand the energy barrier that must be overcome.

In another embodiment, predictive model testing and/or verification may be performed on one or more novel molecular structure datasets intended specifically for validation, while a large collection of other datasets may be reserved specifically for training models in an MLIP environment. In other words, a purpose of a validation dataset may be to determine whether the proposed model shows relevant, pertinent results on a new dataset that may not have been tested and observed yet. As such, any new data that is generated becomes a validation set against which the system may spot check the machine learning model. For example, if a sample of molecular structure datasets contains 100 images, 80 of those images may be allocated specifically for the purposes of training a potential predictive model via MLIP, and the other 20 images held back as the test/validation pool. If the training model derived from the 80 datasets produces a potential predictive model against which the other 20 images yield successful testing and observation results, the model may be considered successful.

In still another embodiment, the performing includes increasing and decreasing a temperature of the one or more atomic structures, and determining an activation energy based on the increasing or decreasing the temperature of the one or more atomic structures. This, in turn, may assist with understanding the stability of the atomic structure (and how prone they are to react and/or have a structural change).

It should be noted that current systems may be limited in predicting atomic forces for atoms whose environment is less observed in the training data. As such, the current disclosure overcomes such limitations by improving the prediction of atomic forces by the introduction of atomic weights while training the MLIP. As such, the models disclosed herein may assist with correctly understanding particle movement and interactions. It is also to be appreciated that, compared to conventional systems, the architecture and methods disclosed herein may output potential predictive models containing fewer errors than conventional systems using the same input data.

In addition, based on the machine learned interatomic potentials mode, updated atomic weights may be outputted. In various embodiments, it may be needed to train a machine learning system to ensure a homogenous and unbiased dataset is used in the calculating (and updating) of the atomic weight. In one embodiment, such homogenous and unbiased focus of the training data for the machine learning system may be based on the at least one factor. As such, in one embodiment, to ensure homogeneous and uniform training of the MLIP, the atomic weights calculated previously may be used. In addition, the training of atomic species with lower forces may be emphasized by weighing the force losses with the inverse of the magnitude of force on the atom, where a modified loss function equation may be used as shown previously.

More illustrative information will now be set forth regarding various optional architectures and uses in which the foregoing method may or may not be implemented, per the desires of the user. It should be strongly noted that the following information is set forth for illustrative purposes and should not be construed as limiting in any manner. Any of the following features may be optionally incorporated with or without the exclusion of other features described. In particular, the method 100 may be shown in greater detail within the methods and architectures discussed hereinbelow.

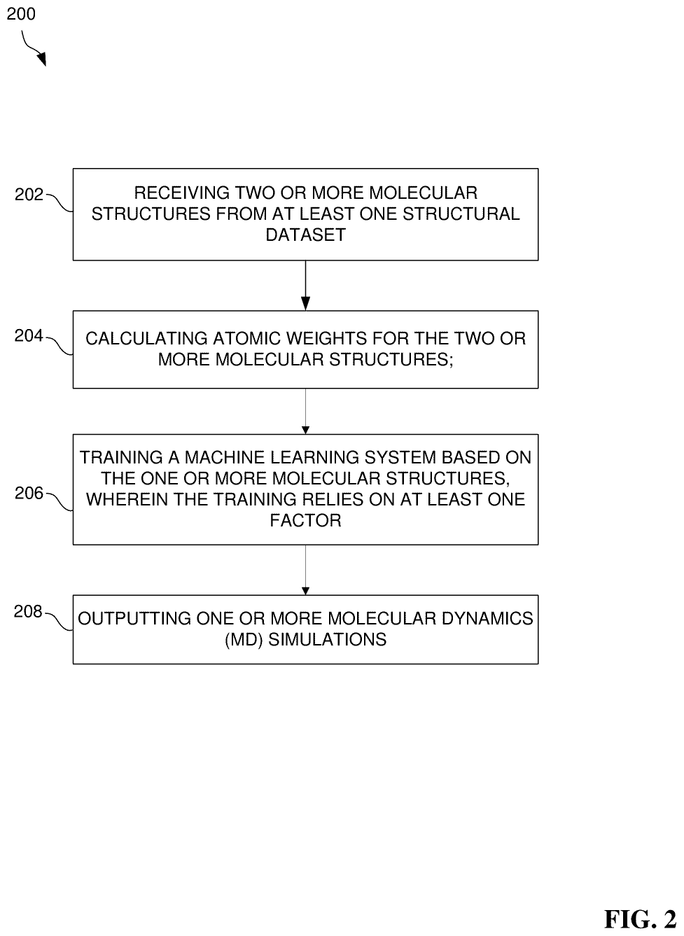

FIG. 2 illustrates a method 200 for predicting ionic conductivity of solid-state electrolytes, in accordance with one embodiment. As an option, the method 200 may be implemented in the context of any one or more of the embodiments set forth in any previous and/or subsequent figure(s) and/or description thereof. Of course, however, the method 200 may be implemented in the context of any desired environment. Further, the aforementioned definitions may equally apply to the description below.

As shown, two or more molecular structures may be received from at least one structural dataset. See operation 202. In one embodiment, the molecular structures that are received from the at least one dataset include at least one of: atomic positions, associated structural energy, atomic weights, or atomic forces affecting one or more atomic structures of the two or more molecular structures. In a more detailed embodiment, the molecular structure datasets may include three key aspects: the structure itself (e.g., the Cartesian coordinates coinciding with the location(s) of the atoms' positions), the energy associated with a given structure, and forces acting on the atoms of the structure. As such, the three elements may function as input parameters used to train a machine learning model. It should be noted that, in so doing, the Cartesian coordinates of the candidate model are not actually used in calculations, but may be converted to mathematical descriptors. For example, even where a given set of atoms' Cartesian coordinates may change, the overall system may essentially remain the same. That is the role the mathematical descriptors play in the system.

As shown, atomic weights may be calculated for the two or more molecular structures. See operation 204. In addition, a machine learning system based on the two or more molecular structures may be trained, wherein the training may rely on at least one factor, and the calculated atomic weights for the two or more molecular structures. See operation 206. In one embodiment, the at least one factor includes at least one of: atomic positions, associated structural energy, atomic weights, or atomic forces affecting the two or more molecular structures.

In another embodiment, the training process may include converting the two or more molecular structures into one or more mathematical descriptors. Additionally, such converting includes using translationally invariant functions and/or rotationally invariant functions. In the context of this present description, a rotationally invariant function may include a function that remains unchanged under rotations. For example, a rotationally invariant function may be observed when the value of the function does not change when arbitrary rotations are applied to one or more pertinent arguments. Further, it should be noted that both radial (constructed as sums of two-body terms) and angular (containing sums of three-body terms) symmetry functions may be employed, which may be comprised of input from different atoms in a molecular structure.

In still another embodiment, the training process may include training a machine learned interatomic potentials (MLIP) model using a traditional and/or diverse ensemble, as discussed hereinabove. In a model training process using machine learning, error estimation capability may additionally be used. Additionally, ensembles may help train on a given dataset multiple times and output multiple outputs for each. Thus, ensemble methods may be thought of as processes that may create multiple potential predictive models, and combine those models to produce a more homogenous result. In known embodiments, ensemble methods, in a machine learning context, may be used to produce more accurate solutions than any single model might.

Further, as discussed herein, a bias-correction for the two or more molecular structures may be applied to the machine learning system. In this manner, ionic conductivity measurements of the two or more molecular structures may be more accurately modeled by MLIP by removing any bias that may be otherwise introduced by the MLIP system.

Additionally, one or more molecular dynamics (MD) simulations may be outputted. See operation 208. In one embodiment, the outputting may include evaluating data from the training with an ensemble of networks to evaluate uncertainty levels against known threshold values, wherein the threshold is a known eV/angstrom value for atomic forces and a known meV/angstrom value for structural energy, and the evaluating of uncertainty levels against known threshold values may indicate a level of uncertainty in ionic conductivity. As such, once initial data is trained using an ensemble of networks, as shown previously, such network may be used to output molecular dynamics (MD) simulations to generate new data. In one embodiment, the outputting of the MD simulations may include performing simulations for the two or more molecular structures.

In another embodiment, the system may further comprise adding one or more new structural datasets to a first database. Additionally, the one or more new structural datasets may be added to a second database that is different from the first database, wherein the one or more new structural datasets in the second database are re-evaluated using a DFT process and are outputted as additional molecular structures in the at least one structural dataset.

In another embodiment, predictive model testing and/or verification may be performed on one or more novel molecular structure datasets intended specifically for validation, while a large collection of other datasets may be reserved specifically for training models in an MLIP environment. In other words, a purpose of a validation dataset may be to determine whether the proposed model shows relevant, pertinent results on a new dataset that may not have been tested and observed yet. As such, any new data that is generated may become a validation set against which the system spot checks the machine learning model.

In still another embodiment, the MD simulations for the two or more molecular structures may include increasing and decreasing a temperature of the one or more atomic structures, and determining an activation energy based on the increasing or decreasing the temperature of the one or more atomic structures.

Further, updated atomic weights based on the performing may be outputted. See operation 208. In one embodiment, the training of the machine learning system may rely upon homogenous and unbiased datasets, which can be incorporate the at least one factor. As such, in one embodiment, to ensure homogeneous and uniform training of the MLIP, the atomic weights calculated previously may be used. In addition, the training of atomic species with lower forces may be emphasized by weighing the force losses with the inverse of the magnitude of force on the atom, where a modified loss function equation may be used as shown previously. Further, for any atom that is not been completely observed, a normalization function may be in place that may ensure full and complete data may be recorded for all atoms within the molecular atomic structures.

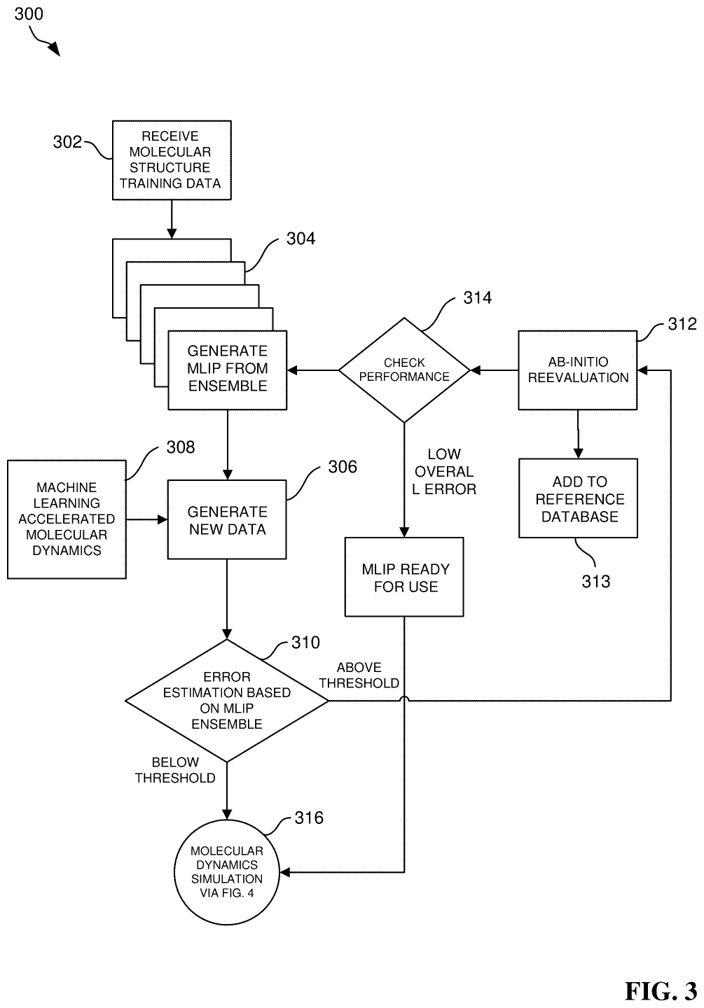

FIG. 3 illustrates a system 300 for automated reiterative machine learned interatomic potentials (MLIP) training, in accordance with one embodiment. As an option, the system 300 may be implemented in the context of any one or more of the embodiments set forth in any previous and/or subsequent figure(s) and/or description thereof. Of course, however, the system 300 may be implemented in the context of any desired environment. Further, the aforementioned definitions may equally apply to the description below.

As shown, the system 300 may receive two or more molecular structures 302 from one or more dataset sources, including but not limited to a first database. For example, as discussed herein, the one or more dataset sources may be derived from DFT. Additionally, a new MLIP model with new data 306 may be generated, where such generation takes place via a network ensemble process (see operation 304) bolstered with optional machine learning accelerated molecular dynamics capabilities 308. In addition, once the MLIP new data 306 has been generated, such data is analyzed and tested to evaluate uncertainty levels (and error estimation) against known threshold values 310, where a threshold may be a known eV/angstrom value for atomic forces or a known me V/angstrom value for structural energy.

If the recorded error estimation value for the given MLIP model tested against known threshold values 310 proves to be above the established threshold (i.e. the error exceeds a maximum amount), the MLIP model may be re-evaluated using ab-initio simulation via DFT process 312, and that tested dataset may then be used to further supplement the structural and/or reference database 313 to be trained again in an iterative manner. Additionally, the tested dataset may also undergo an additional performance verification 314 to determine whether the dataset now falls within threshold guidelines and, if it is determined to be ready for use as an MLIP model, it may continue on via molecular dynamics simulation 316.

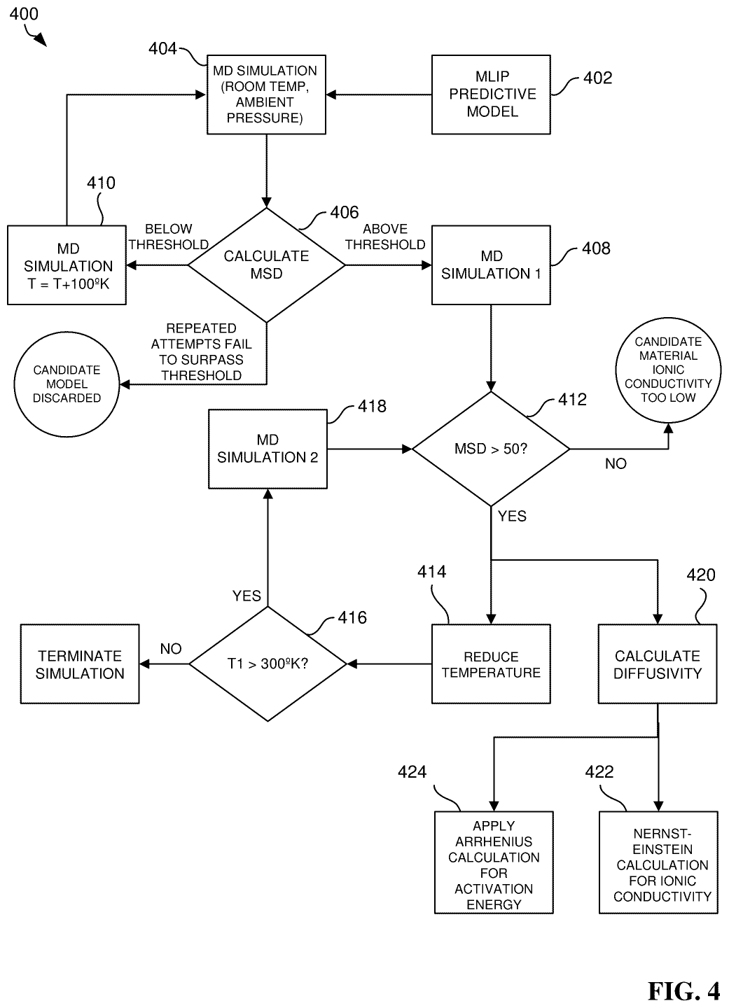

If the recorded error estimation value for the given MLIP model tested against known threshold values 310 proves to be below the established threshold (i.e. it is below a maximum error amount), the model may be further used in the next phase(s) of ionic conductivity maximization via molecular dynamics simulation 316. Further details relating to the molecular dynamics simulation is provided in more detail below with reference to FIG. 4.

FIG. 4 illustrates a system 400 for utilizing MLIP with accelerated molecular dynamics (MD) simulation to calculate the ionic conductivity and activation energy of conductive ion transport in an atomic structure, in accordance with one embodiment. As an option, the system 400 may be implemented in the context of any one or more of the embodiments set forth in any previous and/or subsequent figure(s) and/or description thereof. Of course, however, the system 400 may be implemented in the context of any desired environment. Further, the aforementioned definitions may equally apply to the description below.

As shown, a candidate MLIP predictive model 402 may be passed through molecular dynamic simulation 404, where the analysis takes place, in one embodiment, at room temperature and under ambient atmospheric conditions for 1 nanosecond (and/or a predetermined amount of time), and a mean squared displacement (MSD) 406 may be determined per Eq. 4 provided hereinabove.

If the MSD measurement is observed to be below the 3 Å² threshold, the temperature may be increased 100° K and a further molecular dynamics simulation performed at that increased temperature 410, and the MSD 406 recalculated. It should be noted that this temperature increase-and-test cycle may repeat until the temperature has been increased to, in one embodiment, 800° K. If, at that time, the MSD measurement is still observed to be below the 3 Å² threshold, it is determined that the candidate material structure will not be conductive enough when returned to room temperature and the candidate predictive model may be discarded.

If the MSD measurement is observed to be above the 3 Å² threshold at any time in the observation process (from room temperature up to room temperature plus 800° K), another molecular dynamics simulation 408 may be conducted for 4 nanoseconds and another MSD measurement 412 taken. If, at the higher temperature, the MSD measurement 412 is observed to be lower than 50, the candidate material model may be disregarded (which may be due to the iconic conductivity being too low). If, on the other hand, the MSD measurement 412 is observed to be greater than 50 at that temperature, the system 400 may advance to subsequent calculations.

For example, in one embodiment, following MSD measurement 412, the candidate material structure's diffusivity 420 may be calculated, shown above as Eq. 5. Additionally, the candidate material's ionic conductivity 422 may be calculated using a Nernst-Einstein equation, shown above as Eq. 6. Further, the activation energy for the ionic conduction may be calculated, wherein the testing and observation process may be repeated at lower temperatures (until room temperature) via the Arrhenius equation 424 applied, shown above as Eq. 7.

The above calculations, in various embodiments, can be further supplemented with additional data obtained via step 414. For example, where the MSD measurement 412 is observed to be greater than 50 at a very high temperature, the temperature may be reduced 414 (such as by 100° K and/or by a predetermined amount) and if it is determined that the resulting temperature of the candidate material is less than 300° K, the simulation may be terminated. On the other hand, if the temperature the 300° K, a molecular dynamics simulation 2 418 may be conducted for 4 nanoseconds (and/or any predetermined length of time) and the resulting candidate material may be passed back to the MSD measurement 412 for additional processing. In one embodiment, temperature may be reduced (per 414) such that various MSD readings (at different temperatures) can be obtained to facilitate calculation of the activation barrier using the Arrhenius equation. As such, in particular, the Arrhenius equation 424 may be more easily calculated via additional data (via step 418) at different temperatures (via step 414).

As such, the system 400 may be used to calculate ionic conductivity and activation energy of a candidate material.

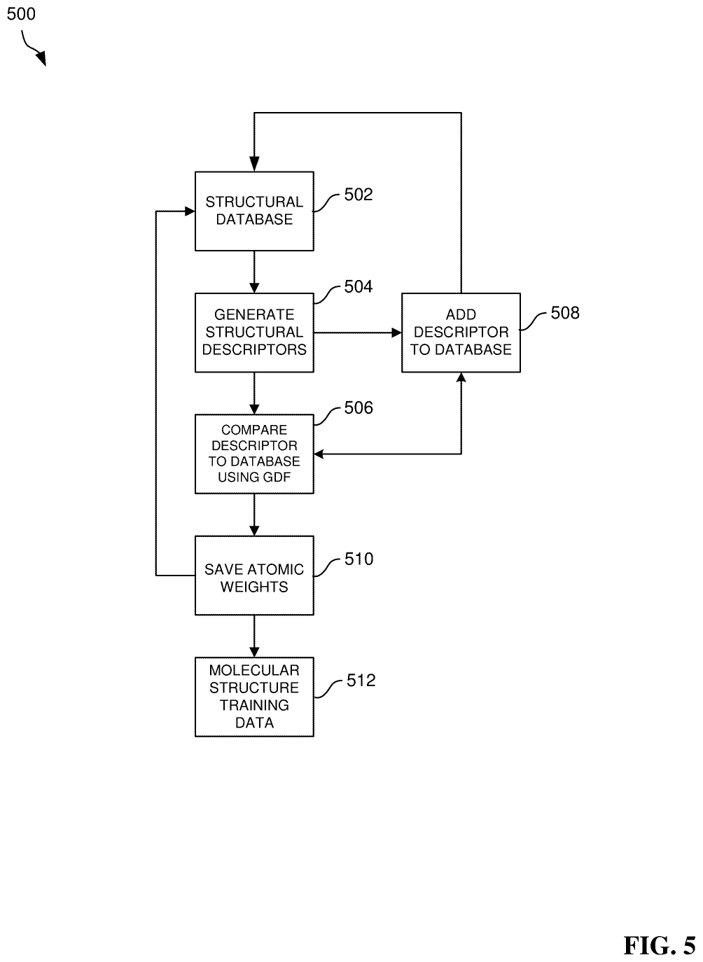

FIG. 5 illustrates a system 500 for developing molecular structure training data, in accordance with one embodiment. As an option, the system 500 may be implemented in the context of any one or more of the embodiments set forth in any previous and/or subsequent figure(s) and/or description thereof. Of course, however, the system 500 may be implemented in the context of any desired environment. Further, the aforementioned definitions may equally apply to the description below.

In one embodiment, molecular structures that are received from the at least one dataset may include initial data obtained using ab-initio simulation via a density functional theory (DFT) process. It should be noted that, in so doing, Cartesian coordinates of the candidate model may be converted to mathematical descriptors. For example, even where a given set of atoms' Cartesian coordinates may change, the overall system may essentially remain the same. As such, the mathematical descriptors may be used in the system 500.

In another embodiment, AIMD may take a snapshot of a molecular structure, run DFT calculations on that structure, and output, as discussed hereinabove, the Cartesian coordinates, energies, and atomic forces for that particular snapshot. Additionally, AIMD may assume a temperature and pressure acting on these systems and may move the atoms around to create a new configuration. This new configuration can be run via DFT to obtain the energy and forces for the new configuration/snapshot. As such, these criteria may then be processed with a set of multiple DFT calculations to get training data.

As shown, a structural database 502 may be the starting point for two or more molecular structures used to train one or more predictive models designed to generate materials (such as, in one embodiment, solid-state battery electrode components) with maximized ionic conductivity. In one embodiment, the structural database 502 may include at least one of: atomic positions, associated structural energy, atomic weights, or atomic forces affecting one or more atomic structures of the two or more molecular structures. In another embodiment, the molecular structure datasets may include three key aspects: the structure itself (e.g., the Cartesian coordinates coinciding with the location(s) of the atoms' positions), the energy associated with a given structure, and forces acting on the atoms of the structure.

Additionally, the three elements may function as input parameters used to train a machine learning model. Additionally, the Cartesian coordinates of the candidate model may be converted to mathematical descriptors. As such, structural descriptors may be generated via operation 504, and the structural descriptors may be converted, such as by, but not limited to, using translationally invariant functions and rotationally invariant functions. In addition, once the mathematical descriptors generated in operation 504 have been created, such descriptors may also become part of a database (see operation 508; and/or a second database as needed), the purpose for which may be to compare local atomic environments to calculate atomic weights.

In addition, the generated structural descriptors (i.e. mathematical descriptors) may then be compared with the entire database (see operation 506) using GDF and those results may be analyzed and recorded. For example, the analysis and recording may be for further enhancing the collection of datasets in the second database (see operation 508) and calculating and recording atomic weights using GDF (see operation 510), consistent with Eq. 1 provided hereinabove.

Further, the three elements (atomic positions, energy, and forces), combined with the newly determined atomic weights, may function as molecular structure training data 512 intended to train one or more predictive models using MLIP functions. It is to be noted that, within the context of the present description, the detailed process immediately above may be used for generating training data to be imported into the system 300 for automated reiterative MLIP training.

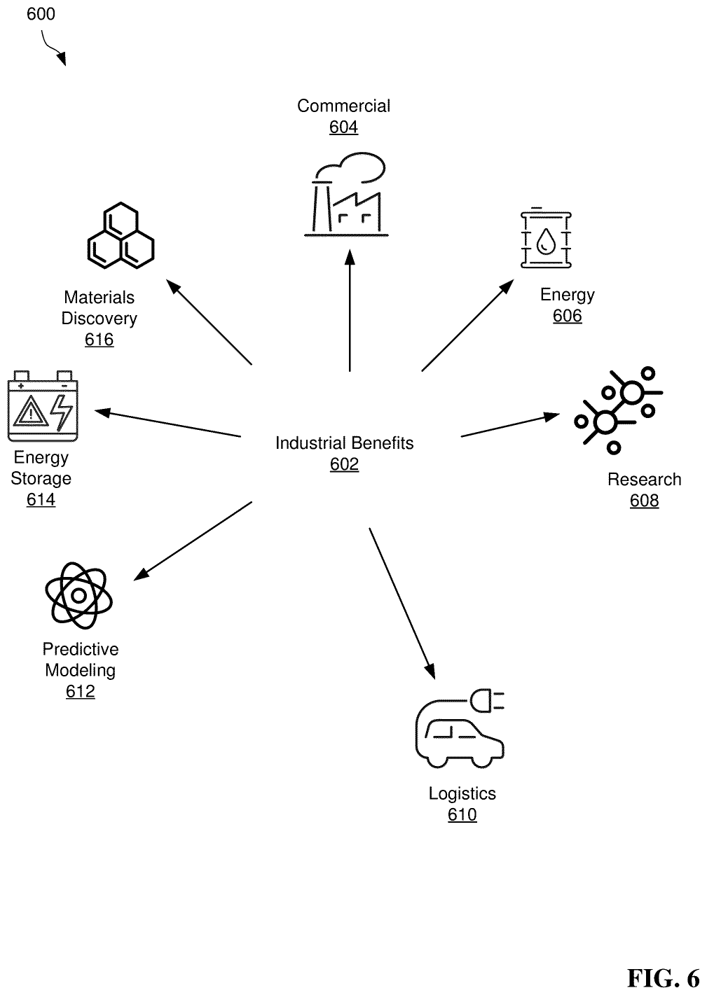

FIG. 6 illustrates a collection 600 of possible industrial benefits of novel atomic structure discovery through machine-learned modeling, in accordance with one embodiment. As an option, the collection 600 may be implemented in the context of any one or more of the embodiments set forth in any previous and/or subsequent figure(s) and/or description thereof. Of course, however, the collection 600 may be implemented in the context of any desired environment. Further, the aforementioned definitions may equally apply to the description below.

As shown, industrial benefits 602 may include commercial 604, energy 606, research 608, logistics 610, predictive modeling 612, energy storage 614, materials discovery 616, etc. It is to be appreciated that any industrial benefit 602 shown is merely exemplary and should not be limiting to the disclosure herein in any manner.

Further, the collection 600 is intended to represent the wide applicability of the present disclosure to a variety of industries. Materials, at a global context level, relates to every human throughout the world. As such, generation of novel materials may affect all industries worldwide.

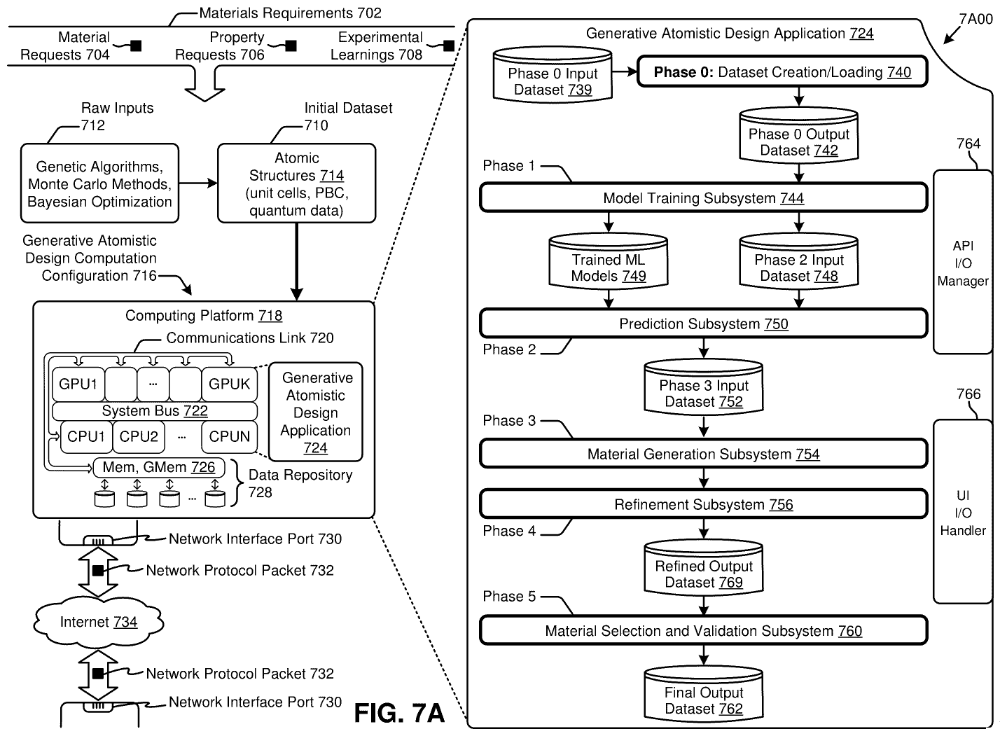

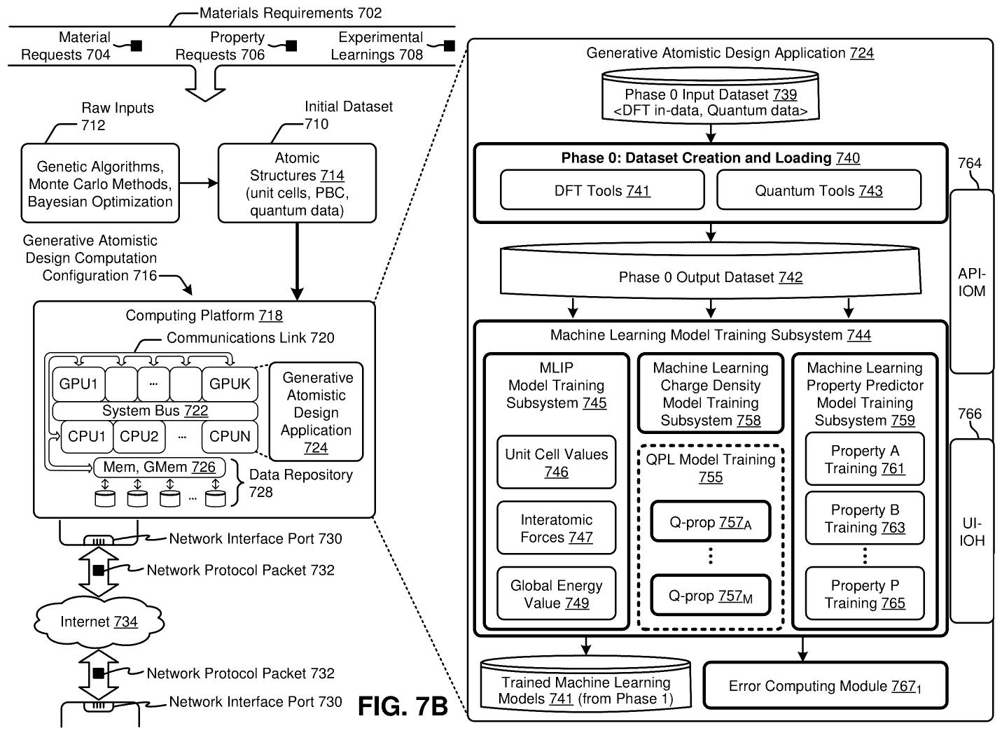

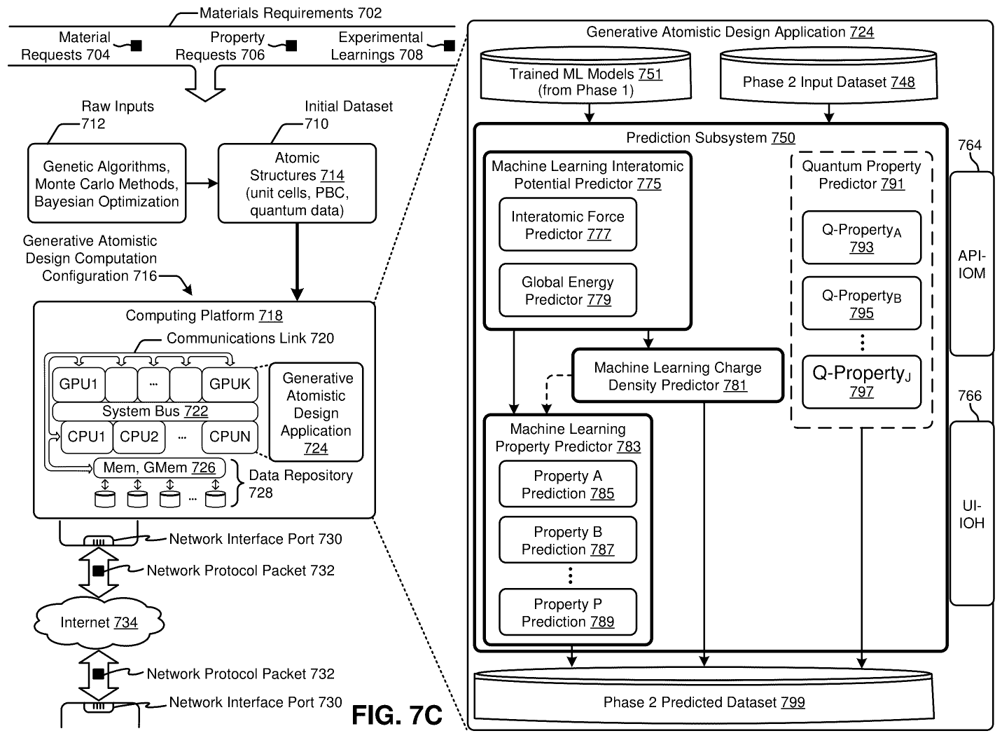

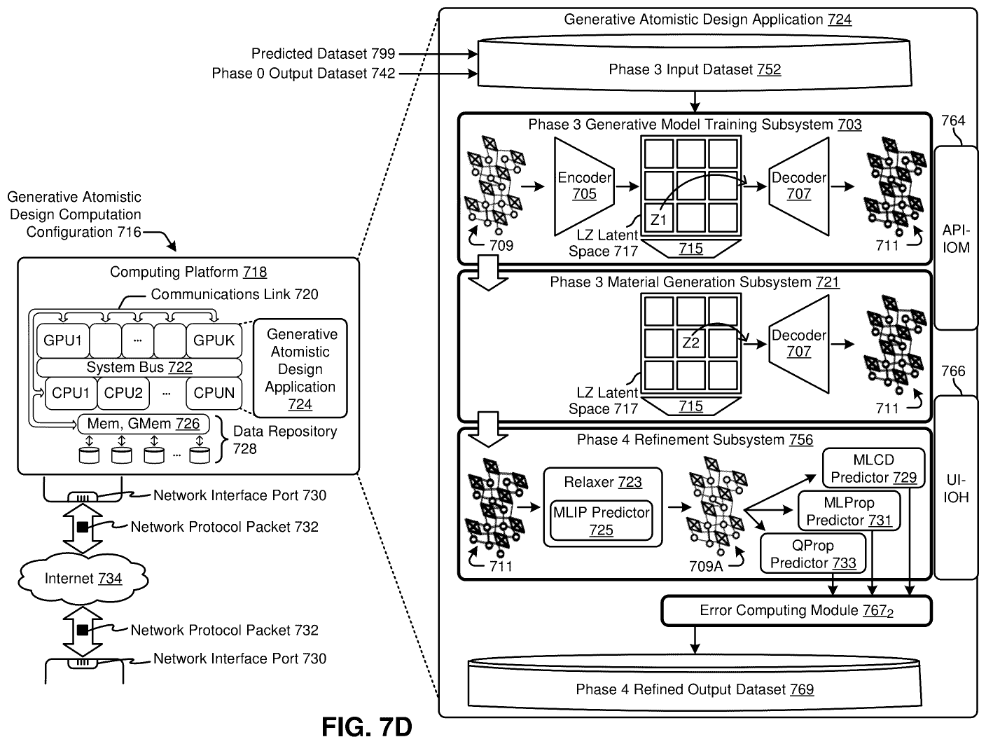

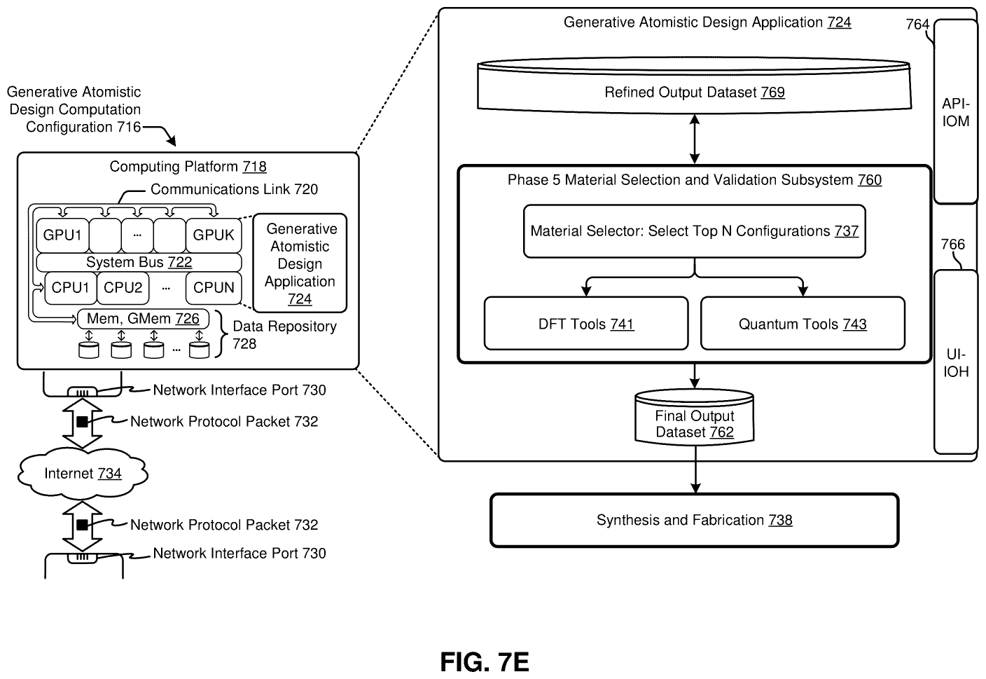

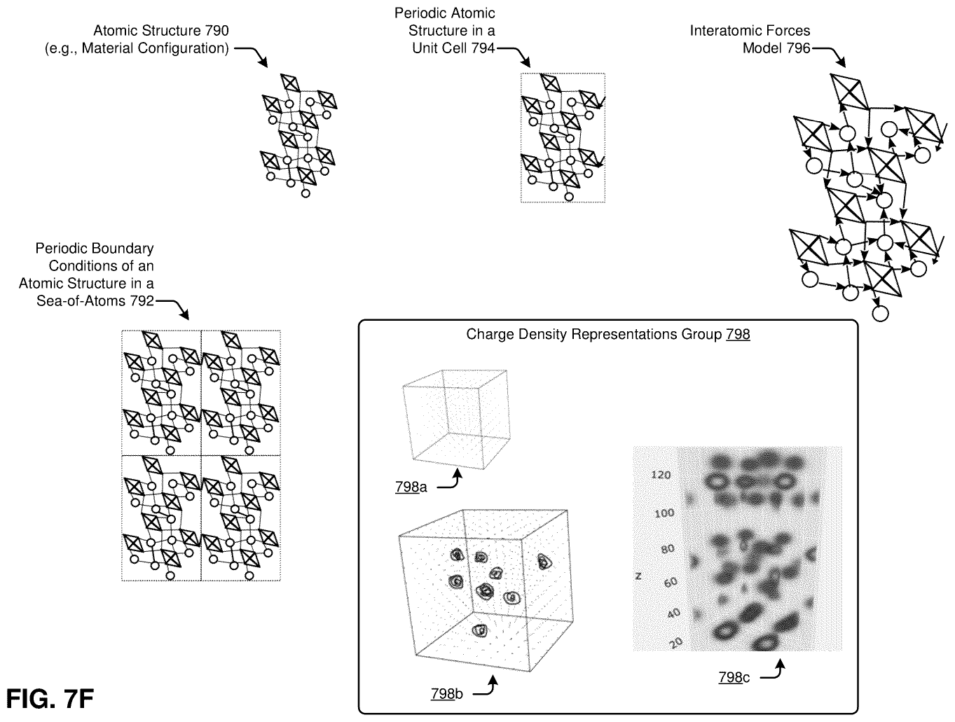

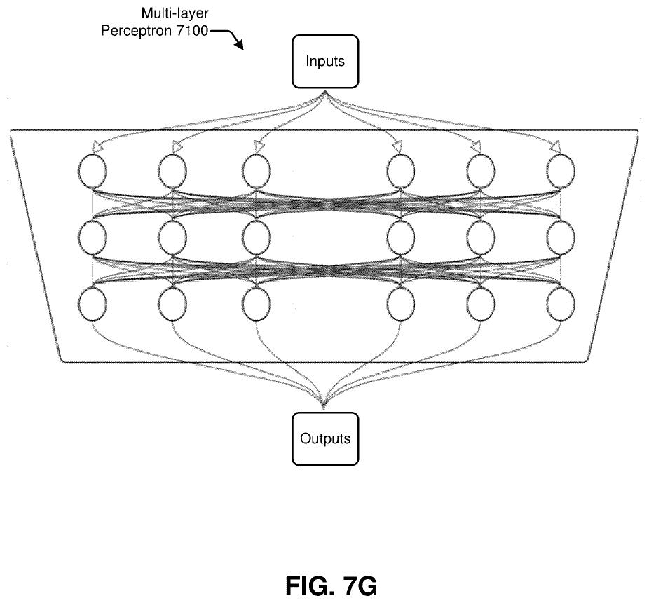

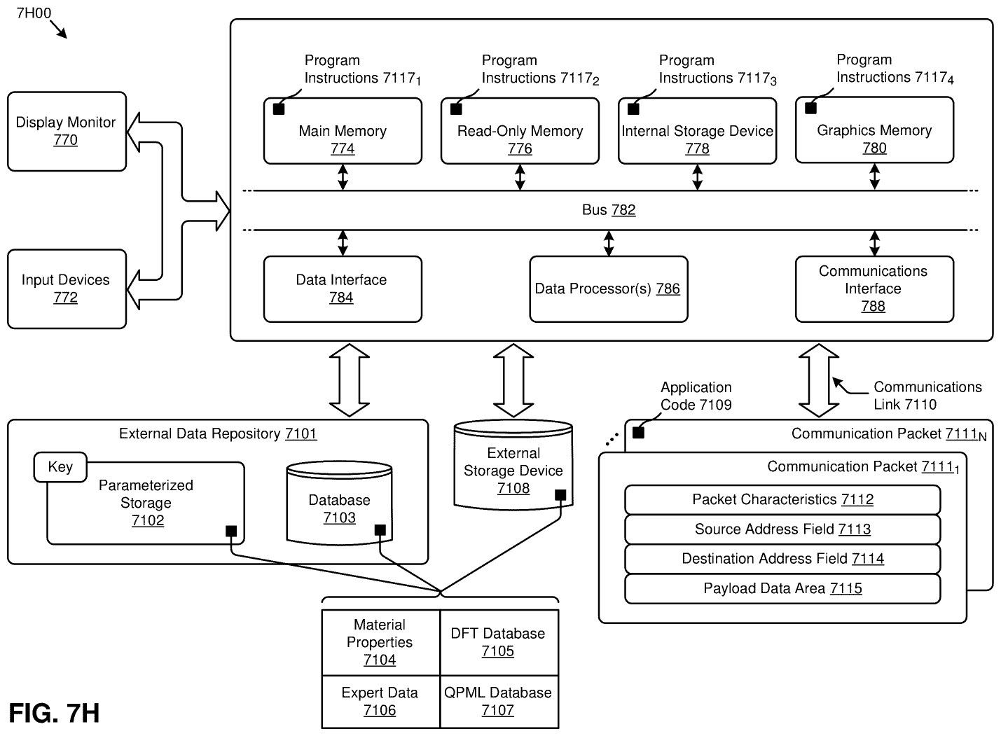

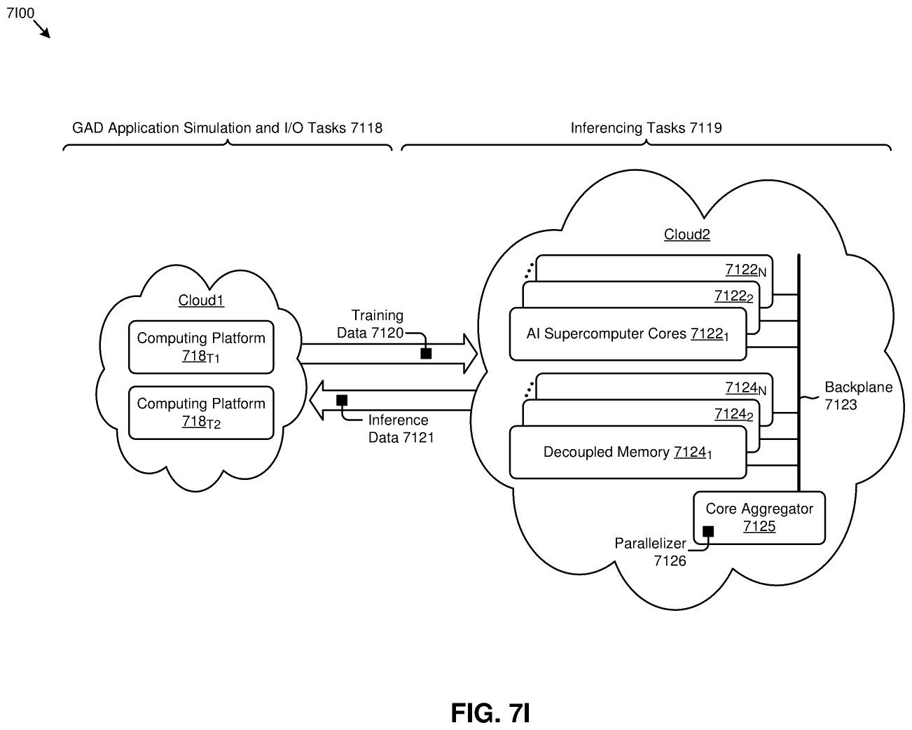

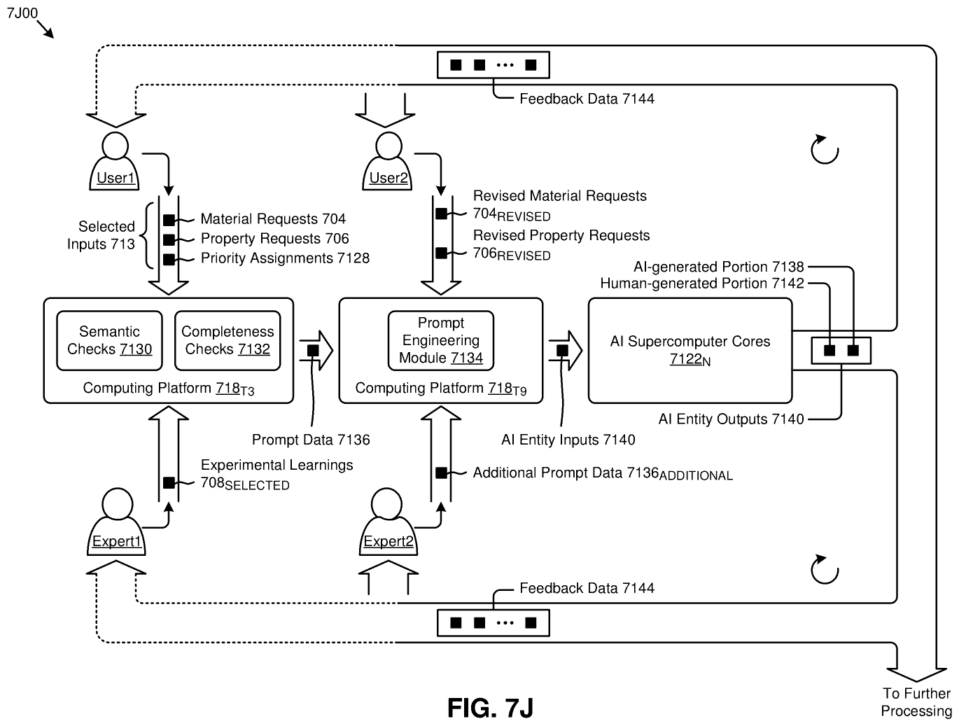

FIG. 7A depicts an environment in which embodiments of the present disclosure can operate. As an option, one or more aspects shown in environment 7A00, including any instancing of the shown components, may be combined to realize one or more of the techniques described herein.

It is to be appreciated that FIGS. 7A-7J may provided further details relating to possible architectures, integration of machine learning systems, and enablement of how such systems may operate. Of course, it is to be appreciated that such Figures may be implemented in any manner within the context of predicting ionic conductivity of materials including solid-state electrolytes.

FIG. 7A discloses a generative atomistic design application (GAD application) for design of materials, where the GAD application may be implemented to execute using a generative atomistic design computation configuration 716 on a computing platform such as 718. Generative atomistic design application 724 (GAD application) for designing materials executes on at least one processing element, which may be a central process unit (CPU) and/or a graphics processing unit (GPU). In some embodiments the generative atomistic design application is further customized to execute on one or more specialized processing elements such as a neuromorphic processor, and/or an array processing unit, and/or a field-programmable gate array (FPGA) or other operational component. Furthermore, generative atomistic design computation configuration 716 itself is not limited to an implementation in any specific semiconductor technology and may be, wholly or in part, implemented using a quantum computing platform or a quantum computer or an opto-electronics based computing platform or a photonics computer or electro-optical computer.

In one embodiment, computing platform 718 comprises processing elements such as one or more CPUs (e.g., CPU1 through CPUN) and/or one or more GPUs (e.g., GPU1 through GPUK), system bus 722, and a processor memory and/or graphics memory 726. In addition to system bus 722, there may also include one or more communication links 720 or a network-on-chip (NOC) links in the computing platform to allow communication between various processing elements and also between the various processing elements and the peripheral interfaces such as connections to external storage devices, external data repository 728, and/or one or more network interface ports 730 optionally coupled to the internet 734 and communicating via one or more packets such as network protocol packet 732 using at least one communication protocol.

The generative atomistic design application 724 may be customized to run on one or more CPUs and/or one or more GPUs depending on the performance needed to achieve a certain computation throughput and the size of the problem being solved. A portion of the GAD application may run on one or more GPUs and another portion of the GAD application may run on one or more CPUs. Yet another portion of the GAD application may run on some specialized processing elements. The GAD application may comprise specialized software subsystems and it may further use some standard middleware or software components (e.g., libraries etc.) and application program interfaces (APIs) for accessing data from storage, etc. In no case, must it be construed that the GAD application contains only standard or open source or other freely available libraries and frameworks only. The GAD application is custom built for the generative design of various materials at the atomistic level with a primary focus on solid-state materials.

The paradigm of using the GAD application starts with one or more users/experts seeking to design one or more materials of interest. Several inputs may be received that comprise materials requirements 702. Materials requirements 702 may further comprise specific materials with specific compositions (chemical and/or physical) in the form of material requests 704 (for example, a solid-state electrolyte, an amorphous ceramic, etc.), and one or more material property requests (e.g., global property or gross property or macro property) 706 (for example, conductivity or ionic conductivity, dielectric constant, dielectric strength, etc.) and is not limited to just these properties. In the context of this disclosure, material requests could be in the form of material classes (for example, pure metals, metal alloys, semiconductors such as silicon; or germanium or compound semiconductors such as gallium arsenide or indium phosphide, polymers such as thermoplastics, thermosetting plastics, elastomers, biopolymers, ceramics such as porcelain, silicon carbide, alumina; or amorphous ceramics such as glass, composites such as fiberglass; or biomaterials such as hydroxyapatite, collagen, biodegradable polymers; or solid-state electrolytes such as beta-alumina, lithium phosphorous oxynitride; or polymer based solid electrolytes such as polyethylene oxide with lithium salt; or piezo electric materials, ferroelectric materials, or smart materials such as nitinol, electrorheological and magnetorheological fluids and gels; or high temperature materials like Inconel and titanium alloys, hybrid materials, photonic materials, amorphous metals and amorphous polymers, etc., but not limited to these alone).

The material classes may also include newer kinds of materials and known material types. Material properties requested may include one or more of several global properties such as electrical conductivity, ionic conductivity, thermal conductivity, hardness, dielectric constant, dielectric strength, corrosion resistance, reactivity, surface tension, electrical resistivity, electric susceptibility, electrostriction, permittivity, piezoelectric constants, Seebeck coefficient, hysteresis, diamagnetism, hall coefficient, magnetostriction, permeability, pyromagnetic coefficient, piezo magnetism, bulk modulus, density, ductility, elasticity, mass diffusivity, specific heat, luminosity, photosensitivity, refractive index, transmittance, photoelasticity, boiling point, melting point, thermal expansion coefficient, etc., but not limited to just these properties. Materials requirements 702 may further comprise experimental learnings 708 in the form of measurement data (e.g., tables, files, records, etc.) such as measurements deriving from surface differential reflectivity (SDR), diffuse reflectance spectroscopy (DRS), diffraction data, interference waveforms, etc. The experimental data is not limited to the properties listed in this disclosure and may, in the future, include newer properties not yet discovered or conceived of as of the filing date of this disclosure. The intended area of exploration will inform what density functional theory (DFT) computations (including any molecular dynamics simulations) or other quantum mechanical computations using techniques such as quantum many-body perturbation models (e.g., random phase approximation (RPA), GW approximation, etc.), density matrix renormalization group, dynamical mean field theory, variational quantum eigensolver (VQE), variational quantum thermalizer (VQT), etc.) will be performed to generate the initial training dataset.

Based on the new material requirements, a material expert may use one or more of available methods and tools (e.g., genetic algorithms, or Monte Carlo methods, Bayesian optimization, etc.) to generate inputs 712 one or more atomic structures (either periodic atomic structures with associated period boundary conditions or non-periodic atomic structures such as molecules), which are appropriate kernels for creating desired structures as a part of initial dataset 710. Certain instances of any initial dataset 710 may further comprise associated quantum data (for example, quantum states, Hamiltonian operators or functions, correlation functions, etc.) corresponding to the atomic structures and properties chosen and which cannot be modeled using DFT techniques. This system only works when trained on an initial dataset that is representative of a material class of interest. Therefore, the system must always start from a set of “known” materials and their associated properties. It can then learn from them and suggest newer configurations that may optimize a property or properties of interest.

In the context of this disclosure, atomic structure (sometimes also known as atomic configuration or material configuration) refers to an arrangement of atoms in matter. Atomic structures may be periodic or non-periodic. Furthermore, this atomic structure may also refer to other atomic scale structures (e.g., molecular structures, structure of ligands, etc.). An atomic structure or atomic configuration may contain information about the atomic species/atomic type and atomic location such as coordinates along with the lattice vectors, which define their periodic boundary conditions (PBCs). In the context of this disclosure, a periodic boundary condition associated with an atomic structure is a simulation artifice to represent a “sea-of-atoms” (e.g., see sea-of-atoms 792 in FIG. 7F), where the atomic structure is imagined to repeat indefinitely in one or more directions. To simulate a finite subset of a sea-of-atoms, a non-periodic atomic structure that repeats may be coupled with a set of boundary conditions in a unit cell (comprising lattice vectors that define periodic boundary conditions of the atomic structure) to create a periodic atomic structure for simulation. The set of boundary conditions (e.g., periodicity of location information, forces, bonds, etc.) are called the periodic boundary conditions (PBCs). The atomic structure (e.g., the unit cell 794 in FIG. 7F) may comprise a non-periodic atomic structure 790 along with the associated periodic boundary conditions. In some embodiments, different representations of atomic structures 714 can equivalently capture the boundary conditions of the same periodic atomic structures.

Atomic structures are foundational in materials science, as a particular atomic structure controls a material's properties, behaviors, and the material's functionalities. Whether it is a simple arrangement like a crystal lattice in metals or a more complex configuration in amorphous materials, the atomic structure is the embodiment from which all global/macroscopic material properties emerge.

The generative atomistic design application 724 in 7A receives several inputs that may comprise initial dataset 710 comprising one or more atomic structures 714, corresponding or associated quantum data, etc. The GAD application is configured to execute in phases. Strictly as an example, a phase 0 might be configured to prepare, create, load, and/or receive one or more models of materials starting from atomic structures 714 (e.g., periodic atomic structures), and/or quantum data, and/or atom types, and/or atom locations, etc., whereas further phases are configured to perform operations as may be required for additional machine learning materials and/or additional model training and/or for making additional predictions or inferences pertaining to any or all of interatomic forces, global energies, interatomic potential, charge densities, global properties, quantum properties, material configuration/atomic structure generation, atomic structure refinement, material selection, etc.

In one embodiment as shown in 7A, the GAD application (e.g., generative atomistic design application 724) comprises subsystems for 5 phases:
Phase 0 is a dataset creation/loading phase implemented using dataset creation/loading subsystem 740, which receives initial dataset 710 that may be taken in as phase 0 input dataset 739, in any appropriate format, and added to any existing instances of phase 0 input datasets. The updated instances of phase 0 input datasets are then used to load and/or create further datasets (for example, phase 0 output dataset in 742) using one or more DFT-based tools for use in other phases.
Phase 1 is a machine learning model training phase implemented using machine learning model training subsystem 744 controlled by a training manager that uses a training dataset (e.g., phase 0 output dataset 742) to train one or more machine learning predictive material models (such as multi-layer perceptron 7100).
Phase 2 is a dataset generation phase (controlled by a dataset prediction manager) using predictive machine learning and implemented using prediction subsystem 750 that uses the material models trained in Phase 1 (e.g., trained machine learning models 749) along with phase 2 input dataset 748 comprising atomic structures, quantum data, etc., for inference/prediction/output of phase 2 datasets (e.g., phase 2 prediction subsystem 750).
Phase 3 is a materials generation phase implemented using material generation subsystem 754 that receives input dataset 752 comprising multiple atomic structures (or atomic structures with PBCs) along with the corresponding/associated charge densities, and/or one or more predicted properties (i.e., property values) associated with each of the corresponding periodic atomic structures. The material generation subsystem trains a generative machine learning model to generate new material configurations comprising unit cells, and/or periodic unit cells, and/or other representations of atomic structures.
Phase 4 is a refinement phase (e.g., refinement subsystem 756) in which the material configurations (atomic structures, unit cells, etc.) generated by a generative machine learning model are consumed by refinement subsystem 756 that refines the generated structures and associated charge density and property values to relax the atomic structures to make them stable by adjusting the locations of atoms so as to reduce interatomic forces inside of them and create refined datasets (e.g., refined output dataset 769).
Phase 5 is a material selection and validation phase that is implemented using material selection and validation subsystem 760, which takes in a refined dataset (e.g., refined output dataset 769) and selects one or more candidate materials and their corresponding/associated data, which are stored in a final output dataset (e.g., final output dataset 762). The material properties and charge densities of the candidate materials may be accurately computed in this phase using the density functional theory tools and models. In addition to the foregoing candidate materials as computed in this phase, other properties and/or any aspect of a material and/or its simulation model counterparts can be stored in final output dataset 762.
Any aspects of any operational element of any of the foregoing phases of computation within the generative atomistic design application can be controlled via a user interface (e.g., user interface handler, UI-IOH, 766), and/or via an application programming interface (e.g., API I/O manager, API-IOM, 764).

Machine learning (ML) is a subset of techniques of artificial intelligence that involves teaching computers to identify patterns and make decisions from data without being explicitly programmed for specific tasks. In the context of this disclosure, and in the context of materials science, machine learning can be employed to predict material properties, to predict material behaviors, and to design new materials. By analyzing precomputed data (e.g., including or based on atomic structures and/or including or based on observed or calculated system dynamics and/or including or based on observed or calculated material properties), machine learning algorithms can efficiently identify relationships that might be absent (or obscured) when using traditional analytical methods. This offers the potential to accelerate the discovery of innovative materials, optimize manufacturing processes, and enhance the understanding of complex material behaviors. Machine learning algorithms may be implemented using any of the generally known methods. However, the customization of some of the generic methods for specialized applications in the context of the GAD application for generation of new materials is merely one of the many subjects of this disclosure.

### Phase 0: Dataset Creation Phase

In order to support the various execution phases, the generative atomistic design application 724 uses several APIs, data structures, and static or constant data and functions, which may be distributed into and/or accessible by several subsystems. One of these subsystems, used in phase 0 of the dataset creation phase, is a density functional theory subsystem that supports density functional theory (DFT) tools and functions (e.g., the shown DFT tools 741). The density functional theory subsystem uses density functional theory as is known in the study of physics, chemistry, or materials science to compute datasets (e.g., phase 0 output dataset 742) that comprise electronic structures and properties of atomic configurations (e.g., atomic structures, charge densities, global energy, interatomic forces, global properties, etc.) The dataset creation phase optionally may also use one or more subsystems for creating or loading quantum properties, models, and functions that optionally provide quantum property models and values to the phase 0 output dataset 742. Phase 0 output dataset 742 and phase 0 input dataset 739 may be used by later phases such as Phase 1 through Phase 5, and/or by their related subsystems (e.g., predictive material model training subsystem 744, prediction subsystem 750, material generation subsystem 754, a refinement subsystem 756 and/or validation subsystem 760), as and when requested.

Density functional theory (DFT) is a computational approach used in physics, chemistry, or material sciences to investigate material properties, and more often, the electronic properties of many-body systems, like atoms, molecules, ligands, ions, and/or solids. Instead of trying to track each electron's movement (or each hole's movement in solid-state materials, for example, semiconductors) or each ion's movement, which can very complex both physically and computationally, DFT focusses on the overall electron density in one such case (e.g., how electrons are distributed in space) and simplifies the problem, which makes it feasible to predict how atoms, molecules, etc. behave in configurations—thereby helping the design of new materials—as well as chemical reactions. Although DFT focusses on simplifying the many-body problem mentioned above, DFT is still computationally very expensive. The present disclosure uses machine learning methods and subsystems to further speed up the vast number of computations needed to design materials at the atomic scale.

One aspect of the GAD application is to use predictive machine learning to speed up computations that would otherwise be done by a density functional theory (DFT) subsystem and/or quantum property calculation methods (e.g., quantum many-body perturbation theory (e.g., RPA, GW), density matrix renormalization group, dynamical mean field theory, variational quantum eigensolver (VQE), and variational quantum thermalizer (VQT), etc.), which are computationally expensive. In the context of this disclosure, predictive machine learning (used in some phases of the GAD application) is a subset of machine learning (e.g., supervised machine learning) where machine learning models (e.g., machine-learned surrogate models) are trained to make predictions about future or unseen data based on patterns identified from historical or known data. The primary goal is to output specific values or classifications based on input features. For instance, in the context of this disclosure, in materials science, predictive machine learning might be used to predict the conductivity, strength, or melting point of a material based on its atomic structure.

One further subsystem used in GAD application includes the in-memory data, functions, and APIs related to one or more models trained on material classes and properties that comprises a set of numeric weight values associated with pairs of “artificial neurons” in one or more layers, which may be selected for further use in the various phases of the generative atomic design application simulation. At least one material class and properties model could be related to material requests 704 and property requests 706.

Some embodiments may train (e.g., for the purpose of quantum property learning (QPL)), quantum property learning model 755. Such learned quantum properties (e.g., quantum property Q-Prop 757A through quantum property Q-Prop 757M) may be included in the GAD application. This QPL model can be used, for example, when a material structure needs to be refined using certain quantum properties.

In order to initiate a workflow, a material class may be selected and at least one associated property to target material generation. In one example embodiment, an inorganic material for use as a battery's solid-state electrolyte with high ionic conductivity may be targeted. In this case, the material class would be “solid-state electrolyte” and an associated property would be “ionic conductivity.” This information is used to inform the use of DFT to create a dataset for use in one or more of the following phases.

The dataset creation phase of the simulation (phase 0 portion of the GAD application simulation) outputs as a part of the dataset creation phase output into (1) one or more representations of atomic structures, (2) one or more atomic species and coordinates, (3) one or more corresponding/associated periodic boundary condition models (if any) comprising lattice vectors, and (4) one or more corresponding/associated interatomic force models. Some or all of the foregoing may include characterization of forces on each individual atom, and/or one or more corresponding global energy values, and/or one or more corresponding volumetric charge density values along with a set of other simulation parameters including any of a variety of properties (e.g., macro or gross properties and/or quantum properties, etc. as may be identified or selected by a user/expert. The dataset creation phase output dataset is used in the machine learning model training phase and/or it may be used in the dataset generation/prediction phase and/or any other phase that may receive it and use it as needed.

### Phase 1: Machine Learning Model Training Phase

In the embodiment shown in FIG. 7B, the machine learning model training phase is used for materials learning, and is implemented using machine learning model training subsystem 744. Machine learning model training subsystem 744 receives the dataset creation phase output (for example, phase 0 output dataset) and, in response to the received dataset, machine learning model training subsystem 744 trains the machine learning model for materials learning.

Machine learning model training subsystem 744 further comprises a machine learned interatomic potentials model training subsystem (e.g., MLIP model training subsystem 745, which is controlled by a training manager. As shown, the MLIP model training subsystem further comprises an atomic structure model training capability, which receives as input one or more atomic structures, unit cells, PBCs, (e.g., unit cell values 746, etc.) from the input dataset (e.g., phase 0 output dataset created by DFT tools), and optionally, by quantum tools 743)), which in turn is used to train one or more periodic atomic structure models inside the MLIP model. In some embodiments, a portion of the machine learned interatomic potentials model may be trained using one or more unit cells and/or periodic atomistic structures as received in an input dataset.

In the context of this disclosure the term an “atomic structure” may refer to a structure of atoms or molecules, along with associated periodic boundary conditions, and is not limited to these alone (e.g., ions, ligands, etc., may also be included).

MLIP model training subsystem 745 may receive as input, one or more periodic boundary conditions (PBCs) along with the associated atomic structures in the input dataset as applicable to the corresponding/associated atomic structures. The atomic structures' model training capability includes consideration of periodic boundary conditions so as to train one or more periodic boundary conditions (e.g., see the illustration of a unit cell 794 in FIG. 7F) that allow simulation to proceed on a limited size atomic structure. The results of such a simulation can be interpreted to repeat, subject to the constraints of any applicable periodic boundary conditions.

In the context of this disclosure, periodic boundary conditions (PBCs) comprise parameters, techniques, tools and methods used in simulations where a system of atoms or molecules, etc., (e.g., a group of atoms) is configured to appear to repeat indefinitely in all directions. By using PBCs, researchers can simulate a small portion of a larger system while effectively mimicking the behavior of a much larger system, or an infinite system. This technique is especially useful in reducing computational costs while still capturing the essence of large-scale phenomena.

The MLIP model training subsystem 745 may further comprise global energy value training capability that is used to train the MLIP model along with the periodic atomic structures. MLIP is trained to model the potential energy surface or the interatomic potential that can be further used to conduct structural optimizations (for example, energy minimization computations) or run long range/larger scale molecular dynamics simulations.

MLIP model training subsystem 745 further comprises interatomic force model training capability 747 that uses at least one interatomic force model in the training phase to compute the interatomic potentials over one or more atomic structures. An illustration to visualize interatomic forces is an interatomic force in a material configuration (e.g., see interatomic force model 796 in FIG. 7F), where the arrows indicate forces exerted on one another by atoms. Interatomic force models are often parameterized on data that ignores ultrashort-ranged interactions and very long-ranged interactions, and the physics of a core wavefunction overlap may not be included in the model. In one explanation of the art, interatomic forces are the forces between the atoms of a molecule and/or an atomic structure due to the electrostatic force between the nuclei and electrons of atoms, and between the nucleus of one atom and the electrons of the other atom or atoms. In some embodiments an interatomic force may be obtained by taking a derivative of the total energy with respect to the atomic coordinates along a path.

In the context of this disclosure machine learned interatomic potentials (MLIP) refers to any method that uses machine learning to model and learn the interactions between atoms in materials. In legacy methods (e.g., DFT-based methods) simulation of atomic interactions require complex and computationally intensive calculations. However, the MLIP methods train models with one or more datasets of known atomic configurations and their corresponding/associated energies and forces. Once trained, the trained models can rapidly predict the behavior of atomic systems under different conditions, making it vastly more efficient, computationally and timewise, than legacy methods. In the context of materials science, MLIP methods can enable simulation of larger systems or longer timescales than are feasible with legacy methods. This allows for a more in-depth exploration of material behaviors, properties, and transformations, leading to accelerated discovery and understanding of novel materials and their related phenomena.

Phase 1 machine learning model training subsystem 744 further comprises a machine learning charge density model training subsystem (MLCD model training subsystem) (e.g., machine learning charge density model training subsystem 758) that receives at least one set of charge density values (to visualize, see illustrations of spatial representation 798a, spatial representation 798b, and graphical simulation data view 798c of the shown charge density representations group 798) that is used by the machine learning charge density model training subsystem to train the machine learning charge density model (MLCD model) with the at least one set of charge density values for a specific material configuration received as input.

The set of charge density values that is received as input is a discretized scalar density field model that spans a volume representing an atomic structure, where the charge density values (shown as dense blobs) are scaler values distributed throughout a unit cell shown as a 3D box with many points distributed throughout. Example graphical simulation data view 798c depicts a 3D view of graphical simulation data of charge density in one embodiment. The charge density values may be in the context of electron charge density, hole charge density, nuclear charge density, ionic charge density, or any charged matter as applicable. The GAD application and the MLCD predictive model can be used in the context of any scalar density field.

In the context of this disclosure, a “machine learning charge density model” refers to a machine learning model trained to predict the charge density associated with a specific material configuration. In this context charge density is a discretized scalar density field that spans the volume represented in the associated atomic structure/material configuration and is represented by a set of charge density values. In other words, charge density refers to the distribution of an electric charge over a certain volume or surface. It measures how much electric charge is packed into a given space. In materials science, particularly when discussing atoms and molecules, charge density provides insights into the electron distribution around atomic nuclei. A detailed determination of charge density is crucial, as it directly impacts the material's electronic properties, chemical reactivity, and bonding characteristics. By modeling charge density, an accurate prediction of atomic and/or molecular interactions, bonds, and the global (e.g., gross and macroscopic) properties of materials can be made.

Phase 1 machine learning model training subsystem 744 further comprises a machine learning property predictor model training subsystem (e.g., machine learning property predictor model training subsystem 759). The machine learning property training subsystem receives at least one material property model and trains an internal predictive model on the specific material property/properties selected. The machine learning property model is trained to predict a scalar value of a global property associated with the entire material configuration (including the atomic structure) associated with it. As discussed hereinabove, the global property examples include band gap, electrical conductivity, formation energy, etc., and is not limited to these alone. In the embodiment of FIG. 7B, examples of global properties of materials under training comprise Property A training 761, and Property B training 763 through Property P training 765, where for example property training could pertain to an electrical conductivity or a band gap value or any one of a number of properties of a material.

In some embodiments, the model training subsystem may optionally include a quantum property learning (QPL) model training subsystem such as 755 that is a machine learning model that trains one or more quantum property models to learn other quantum properties (e.g., quantum property Q-prop 757A through quantum property Q-Prop 757M), which may include quantum properties such as bipartite entanglement spectrum, thermal density matrix, ground state energy of a Hamilton operators or functions, spin expectation values, etc. In one embodiment, quantum property learning may be implemented using variational quantum thermalizer (VQT) as is known in the art.

The generative atomistic design application model also comprises an instance of error computing module 7671, which is a part of this active learning framework where model errors and uncertainty estimates are computed at the end of a training phase and/or a training epoch/regime, and a decision is made as to whether or not a newly evaluated structure must be recomputed with DFT using DFT tools 741. If the errors and uncertainties exceed a selected threshold, the structure will be passed back to the DFT subsystem. This feedback mechanism is used to enhance the predictive machine learning models (e.g., models in the form of one or more atomic structures, models in the form of periodic boundary conditions, etc.). Further, this feedback mechanism is used to enhance or confirm any one or more of, corresponding/associated global energy values, corresponding/associated interatomic force models, corresponding/associated charge density values, and/or corresponding global property values.

Following this approach, the machine learning models are trained to sufficient accuracy with fewer expensive DFT computations. To compute predictive model errors, the shown instance of error computing module 7671 compares machine learned interatomic potentials model training subsystem outputs (e.g., from MLIP model training subsystem 745), machine learning charge density model outputs (e.g., from machine learning charge density model training subsystem 758), and machine learning property predictor model training outputs (e.g., from machine learning property predictor model training subsystem 759) with their corresponding ground truth values generated by the DFT tools and functions subsystem. In this case, the shown instance of error computing module computes errors by comparing the predicted quantum property values from quantum property learning models with generated quantum property values (e.g., from the phase 0 output dataset generated by operation of quantum tools 743. Once model training converges based on a threshold for error, which is either set by a user/expert or by other means (e.g., self-consistently determined within the active learning cycle), the error is assessed, and if the performance is not satisfactory (e.g., where the error breaches a predetermined error-level threshold), phase 0 is repeated with additional phase 0 input and more data is added to phase 0 output dataset 742. Thereafter, the training processes of Phase 2 are repeated.

In some embodiments, criteria for training convergence may be set even when, after the learning rate is sufficiently low, no further improvement in the error is seen. This process is continued until the computed error, including uncertainty estimates, breaches the selected threshold. One method of determining model error is to use a mean-square error computation and minimize the computed mean-square error between a predicted value from a predictive model and an expected value obtained from the determined ground truth. Typically, model uncertainties may be computed in terms of mean and standard deviation computations, and a chi-squared distribution may be used to compute a goodness value associated with the model uncertainties to compare with a threshold. It is also possible to obtain uncertainty estimates using other statistical methods.

The output of Phase 1 (such as a machine learning model training phase) is various sets of machine learning material models of atomic structures, associated periodic boundary conditions, associated global energy predictive models, associated interatomic force models, associated charge density values, and one or more associated global property value predictive models. These various sets of machine learning material models are then used in other phases to perform various tasks/steps including inference/prediction, relaxation, estimation, refinement, and/or other tasks/steps not listed here.

### Phase 2: Dataset Generation Phase

In some embodiments, such as the embodiment shown in FIG. 7C, the generative atomistic design application model (GAD application) includes Phase 2 in the execution/simulation regime, which is a dataset generation phase that is implemented using prediction subsystem 750. In the dataset generation phase, predictive machine learning is used to predict and/or infer datasets for generative machine learning that follows.

Prediction subsystem 750 comprises machine learned interatomic potentials prediction 775, which comprises a capability to receive one or more atomic structures and one or more corresponding/associated periodic boundary conditions, MLIP predictive models, MLCD predictive models, machine learning proportionality (MLProp) predictive models, and/or any other predictive models included in trained predictive models 751. The MLIP predictive model includes the capability to predict interatomic forces as is shown by interatomic force predictor 777, and further includes the capability to predict global energy values as is shown by global energy predictor 779. In various embodiments, the predictive models may be based on various neural network models such as graph neural networks, convolutional neural networks, transformers, tensor field networks, multi-layer perceptrons (e.g., as shown by multi-layer perceptron 7100 in FIG. 7G), etc.

Prediction subsystem 750 may also comprise machine learning charge density predictor 781 that receives one or more predicted and/or learned structures from Phase 1, and uses machine learning inference to infer or predict corresponding/associated charge density values.

In some embodiments, prediction subsystem 750 may also comprise machine learning property predictor 783 that uses previously trained global property models (or macro property models) that predict values of one or more global properties (e.g., property A prediction 785, property B prediction 787, and property C prediction 789).

The outputs of the prediction subsystem are stored in memory in a data structure such as phase 2 predicted dataset 799, which may be forwarded to other subsystems or subroutines or functions or capabilities in various phases of the GAD application.

In some embodiments, quantum property learning predictive model (e.g., quantum property predictor 791) and/or any model trained in a previous training phases may also be used to predict quantum property values (e.g., quantum property values Q-Property_{A} 793 and quantum property value Q-Property_{B} 795 and quantum property value Q-Property_{J} 797), which may be further used to provide higher fidelity global property values where and when feasible.

### Phase 3: Material Generation Phase

In the context of a generative atomistic design application (a GAD application), “material generation and refinement” is a term used to refer to learning and generation of newer atomic structures (or molecular structures or material configurations). In one embodiment, such as is shown in FIG. 7D, Phase 3 of the GAD application is a material generation phase is implemented using a generative model training subsystem (e.g., generative model training subsystem 703), a portion of which also serves the function of material generation (e.g., as implemented by material generation subsystem 721). In one embodiment, the generative model training subsystem 703 and/or material generation subsystem 721 uses generative machine learning, and is trained to learn and create material configurations (e.g., atomic structures, molecular structures etc.) that may yield new materials with optimized properties. The generative model training subsystem 703 and/or material generation subsystem 721 may be implemented using any known or new generative machine learning models.

In the embodiment shown in FIG. 7D, generative model training subsystem 703 is implemented using an auto encoder (AE) unit or variational auto encoder (VAE) unit. The generative model may be implemented using hybrid auto encoders with diffusion, or hybrid variational auto encoders with diffusion, generative adversarial networks (GANs), or any number of other variations. The generative model is trained to reconstruct the input structure and charge density using mean squared error (MSE). The Kullback-Leibler divergence (e.g., KL divergence) is the final loss term. This causes the model to create a latent space with zero mean and unit variance, which facilitates sampling.

The AE unit or VAE unit in generative model training subsystem 703 may be implemented wholly in software or partially in software and partially in hardware or substantially in hardware that is configurable under software control. The AE/VAE unit trains on a material dataset that may be generated by another phase such as the dataset generation phase. The AE/VAE unit implemented using encoder-decoder architecture comprises material encoder 705, data-structure 715 for storing one or more latent vectors (e.g., latent vector Z1) in an LZ latent space 717, a property predictor and material decoder 707 that generates candidate material configurations such as material configuration 711 (which may be, for example, a candidate periodic atomistic structure in a representation as a unit cell, and/or in a representation involving atomic structures with PBC, molecular structures, etc.) along with the associated charge density values and global property values, which are stored in a data structure.

The material encoder, the property predictor, and the material decoder may have the same or a different number of training epochs. Generally, the material encoder, the property predictor, and the material decoder (e.g., material generator) are trained together in a sequence until a convergence is achieved using an input such as atomic structure 709 and some associated property/properties value. (Note that there may be variations in the exact manner of training between different embodiments, and one single method is not mandated.) Additionally, it is to be appreciated that the atomic structure 709 includes a trained dataset of atomic structures, and that the atomic structure 709A includes a refined dataset of atomic structures.

Any/all of the foregoing predictors may be implemented using a network of predictive multi-layer perceptrons (e.g., multi-layer perceptron 7100) or any other predictive network such as a graph neural network etc., that can perform inferencing and/or make predictions that are portions of or derive from training data. In some cases, a machine learning property predictor can be configured to predict property values using mean squared error methods.

In the material generation portion of Phase 3, the material generation subsystems comprising a previously trained instance of material decoder 707 is provided a latent vector (such as latent vector Z2) as input, and the decoder generates a material configuration (periodic or non-periodic atomic structure, molecular structure etc.) as output. Of note, the generated instance of material configuration 711 may not be stable. Also, as the latent vector Z2 changes, so does the generated material. In one embodiment, a latent vector such as latent vector Z1 or latent vector Z2 in the latent space LZ may be implemented using an array of 64 values. The size “64” of a latent vector may be chosen arbitrarily or may be chosen based on experimentation, and without loss of generality, can be any whole number value that is computationally reasonable and can be used to represent the vast space of material configurations that can be generated. The latent vector Z2 is sampled from the latent space LZ using a selected method (e.g., random sampling, neighborhood sampling, etc.). Latent vector Z2 is passed to the trained decoder, which transforms the latent vector into an atomic structure/material configuration as the case may be. The generated atomic structure/material configuration is then passed to a refinement phase.

Generated candidate material configurations may not be “stable” and may have unbalanced interatomic forces that may render a real-world material with such a material configuration (e.g., atomic structure, molecular structure, etc.) as to be unstable and prone to disintegration/decomposition. Therefore, unstable material configurations-such as non-periodic atomic structures, periodic atomic structures, or molecular structures that are deemed unstable-must be structurally relaxed or discarded so they do not disintegrate/decompose.

### Phase 4: Refinement Phase

Material configurations (periodic or non-periodic atomic structures, and molecular structures, etc.) generated in a material generation phase (e.g., in Phase 3) may have very high and/or unbalanced interatomic forces and/or global energy values, which may render such material configurations unstable in the real world. To obtain stable materials with desired properties, such unstable material configurations must be relaxed, and this relaxation is done by refinement subsystem 756. As shown, refinement subsystem 756 includes an instance of a relaxer 723 that in turn comprises a machine learned interatomic potentials predictor (e.g., MLIP predictor 725). The relaxer implements any one or more types of relaxation processes such as simulated annealing or other mechanisms that relocates the atoms slowly to simulate time-varying interatomic forces. This mechanism could be, in principle, any local or global optimization algorithm, gradient based or gradient-free. Typically, in various embodiments of these applications, they would be local, gradient-based optimization algorithms (e.g., the Broyden-Fletcher-Goldfarb-Shanno algorithm (BFGS algorithm), the fast inertial relaxation engine (FIRE), conjugate-gradient method, and/or quasi-Newton methods, etc.).

A machine learned interatomic potentials predictive model may be used to quickly determine the values of the interatomic forces on relaxed material configurations during the relation process. Further, a machine learning charge density predictor such as MLCD predictor 729, a machine learning property predictor such as MLProp predictor 731, along with a quantum property predictor such as qProp predictor 733 may be used, singly or in combination, to determine the charge density profile, the global property values, and the quantum property values of a relaxed material configuration. This may cause the relaxer to adjust the position of one or more atoms in a generated structure to further reduce the interatomic forces on the atoms in the structure until a stable material configuration is obtained.

In one embodiment, the atoms and/or molecules, when initially placed in a simulated environment, may not be in their lowest energy position. In operation, relaxer 723 adjusts the positions of the one or more atoms in response to the forces predicted by MLIP predictor 725. The process of relaxing a structure (e.g., structure relaxer 790) allows the atoms and/or molecules to settle into their locally lowest energy configurations, which often correspond to how they would naturally arrange themselves in reality in the physical world. The processes of FIG. 7D may be repeated by feeding the relaxed structure to MLIP predictor 725 and continuing the process of relaxation until the maximum force on any atom is below a stable maximum force threshold value, which may be set by a user/expert. For example, an acceptable stable maximum force threshold value Fmax may be in the range FX £ Fmax £ FY for a given material under consideration.

Machine learning charge density predictor 781 is used to predict the charge density of a relaxed configuration of an atomic structure at any point during the simulation. A machine learning property predictor (e.g., MLProp predictor 783) is used to predict one or more property value predictions (e.g., property A prediction 785, property B prediction 787, property C prediction 789) associated with the relaxed configuration of atomic structure 709. The extensive use of machine learning based inference/prediction significantly reduces the need to use DFT-based calculations, which can be one or more orders of magnitude computationally expensive.

After the generation of a material configuration, the goodness estimate for the result is computed in terms of a model error and/or model uncertainty estimates. Specifically, in Phase 4, model error is computed as mean squared error taken as a square of the difference between the phase 4 predicted values after the refinement steps and the expected values from the raw outputs (e.g., atomic structures, charge density values, property values, etc.) of the generative model.

To compute predictive model errors, the shown instance of error computing module 7672 compares machine learning charge density model values (e.g., from MLCD predictor 729, ML property model values (e.g., from MLProp predictor 731),), and quantum property values (e.g., from Qprop predictor 733) with their corresponding ground truth values generated by the DFT tools and functions subsystem.

A desired threshold is chosen by simulation/experimentation within a reasonable range, and if a computed uncertainty and/or model error for a candidate material configuration (periodic or non-periodic atomic or molecular structures) is above the chosen threshold, then the candidate unit cell/atomic structure is passed back to the DFT subsystem in phase 0 to recompute accurate energy values, interatomic forces, charge density, and one or more property values accurately. The computed energy (e.g., global energy value), interatomic forces, charge density, and the one or more property values are used to further train the MLIP models, the MLCD models, and the machine learning property models in Phase 1. If the model error for a candidate material configuration (e.g., atomic structure) is below the chosen threshold, then the model is promoted to be included into refined output dataset 768.

### Phase 5: Material Selection and Validation Phase

In the embodiment of FIG. 7E, generative atomistic design application 724 (GAD application) and refined output dataset 769 hold material configurations and/or atomic structures and their corresponding/associated interatomic potential, charge density, and generated energy structures generated by material generation subsystem 721 in the material generation phase (e.g., in Phase 3), which had been subsequently refined by refinement subsystem 756. Refined output dataset 769 is queried to determine the best M candidate material configurations (e.g., the shown top N configurations 737) that are predicted to exhibit the property values in a desired range. The best N candidate material configurations can be based on a goodness factor for comparing any number of candidate material configurations. In some embodiments, <n> is an integer provided by a user/expert. For example, in one embodiment, if materials that minimize formation energy are designed, where n might be desired to be 20, 20 materials may be drawn from the phase 3 dataset based on whichever of those 20 materials have the lowest predicted formation energy. These selected materials are passed to DFT tools 741 and/or to quantum models for validation (e.g., by way of generating their formation energy or by way of confirming material properties). Now, with an understanding that there is a limited number of best candidates to be selected and promoted to undergo accurate simulation using DFT tools, it can be seen that this technique is many orders of magnitude more efficient than alternative approaches (e.g., where a nearly unlimited number of candidates of unknown promise are subjected to further simulation).

The set of atomic structures (e.g., unit cells, periodic atomic structures, material configurations, etc.) that are considered to be best for one or more macro properties, are placed in the final output dataset 762 along with their associated charge density values, interatomic forces, global energy values, and global properties. Characteristics of the foregoing materials are known to be accurate, at least in that they have computed to the accuracy level of DFT tools 741. Further, foregoing quantum tools 743 are used to calculate and/or validate various quantum properties of selected materials.

In some embodiments, one or more atomic structures/material configurations in final output dataset 762 may be used to synthesize or fabricate a physical material (e.g., via synthesis and fabrication process 738).

FIG. 7G depicts a multi-layer perceptron. Any known technique can be used to establish and assign values to individual neurons of a perceptron. As used herein a multilayer perceptron (MLP) refers to a feedforward artificial neural network consisting of interconnected neurons, An MLP can implement nonlinear activation functions. Example embodiments are organized into at least three layers (e.g., as shown in FIG. 7G).

FIG. 7H is a block diagram of an instance of a computer system 7H00 suitable for implementing embodiments of the present disclosure. Computer system 7H00 includes bus 782 or other communication mechanism for communicating information. The bus interconnects subsystems and devices such as a central processing unit (CPU), or a multi-core CPU (e.g., data processor 786), a system memory (e.g., main memory 774, or an area of random access memory (RAM) implemented in any technology), a non-volatile memory in any technology such as FLASH memory or phase change memory or a non-volatile storage device or non-volatile storage area (e.g., read-only memory 776), a graphics memory (e.g., graphics memory 778), internal storage device 780 or external storage device 7108 (e.g., magnetic or optical or photonic or quantum electronic or ferroelectric or semiconductor or phase change memory), data interface 784, and communications interface 788 (e.g., PHY, MAC, Ethernet interface, modem, etc.). The aforementioned components are shown within a processing element partition, however other partitions are possible.

Computer system 7H00 further comprises display 770 (e.g., CRT or LCD or OLED or 3D holographic display), various input devices 772 (e.g., keyboard, cursor control, camera, light pen, etc.), and an external data repository 7101.

According to an embodiment of the disclosure, computer system 7H00 performs specific operations by data processor 786 executing one or more sequences of one or more program instructions contained in a memory. Such instructions (e.g., program instructions 7117₁, program instructions 7117₂, program instructions 7117₃, and/or program instructions 7117₄, etc.) can be contained in or can be read into a storage location or memory from any computer readable/usable storage medium such as a static storage device or a hard drive. The sequences can be organized to be accessed by one or more processing entities configured to execute a single process or configured to execute multiple concurrent processes/tasks/threads to perform work. A processing entity can be hardware-based (e.g., involving one or more cores of homogenous or heterogenous processing elements) or software-based, and/or can be formed using a combination of hardware and software that implements logic and/or can carry out computations and/or processing steps using one or more processes and/or one or more tasks and/or one or more threads or any combination thereof.

According to an embodiment of the disclosure, computer system 7H00 performs specific networking operations using one or more instances of communications interface 788. Instances of communications interface 788 may comprise one or more networking ports that are configurable (e.g., pertaining to speed, protocol, physical layer characteristics, media access characteristics, etc.), and any particular instance of communications interface 788 or port thereto can be configured differently from any other particular instance. Portions of a communication protocol can be carried out in whole or in part by any instance of communications interface 788, and data (e.g., packets, data structures, bit fields, etc.) can be positioned in storage locations within communications interface 788, or within system memory, and such data can be accessed (e.g., using random access addressing, or using direct memory access DMA, etc.) by devices such as data processor 786.

Communications link 7110 can be configured to transmit (e.g., send, receive, signal, etc.) any type of communications packets (e.g., communication packet 7111₁, . . . , communication packet 7111_{N}) comprising any organization of data items. The data items can comprise payload data area 7115, destination address 7114 (e.g., a destination IP address), source address 7113 (e.g., a source IP address), and can include various encodings or formatting of bit fields to populate packet characteristics 7112. In some cases, the packet characteristics include a version identifier, a packet or payload length, a traffic class, a flow label, etc. In some cases, payload data area 7115 comprises a data structure that is encoded and/or formatted to fit into byte or word boundaries of the packet.

In some embodiments, hard-wired circuitry (or hardware) may be used in place of or in combination with software instructions to implement aspects of the disclosure. Thus, embodiments of the disclosure are not limited to any specific combination of hardware circuitry and/or software (program instructions). In embodiments, the term “logic” shall mean any combination of software or hardware including but not limited to a microcode implementation or a programmable gate array (PGA/FPGA) implementation that are used to implement all or part of the disclosure.

The term “computer readable medium” or “computer usable medium” as used herein refers to any medium that participates in providing instructions to data processor 786 for execution. Such a medium may take many forms, including but not limited to, non-volatile media and volatile media. Non-volatile media includes, for example, optical or magnetic disks such as hard drives or disk drives or tape drives. Volatile media includes dynamic or static memory such as RAM or a register file or a sequential memory or even a phase-change memory.

Common forms of computer readable media include, for example, floppy disk, flexible disk, hard disk, magnetic tape, or any other magnetic medium; CD-ROM or any other optical medium; punch cards, paper tape, or any other physical medium with patterns of holes or micro-dots of phase altered materials; RAM, PROM, EPROM, FLASH-EPROM, phase-change memory or any other memory chip or cartridge; or any other non-transitory computer readable medium. Such data can be stored, for example, in any form of external data repository 7101, which in turn can be formatted into any one or more storage areas, and which can comprise parameterized storage 7102 accessible by a key (e.g., filename, table name, block address, offset address, etc.).

In the context of this disclosure a database comprising material classes and their properties, structures, and functional models of density functional theory-based implementations and data, experimental data from materials testing and validation, quantum properties data (e.g., quantum states, Hamiltonian operators or functions, correlation functions, etc.), quantum property learning functions and models, (e.g., quantum machine learning data, functions, and models), and various other simulation parameters may be stored on external data repository 7101 and/or a portion of the database may be stored on external storage device 7108. Further, a portion of the database may be stored on parameterized storage 7102 and furthermore, a portion of the database may be stored in or on database server 7103, and/or in or on any one or more external storage devices. Strictly as examples, any one or more of material properties 7104, DFT database 7105, expert data 7106, and/or Qt modeling language (QPML) database 7107 can be stored in parameterized storage.

Execution of the sequences of instructions to practice certain embodiments of the disclosure are performed by a single instance of computer system 7H00. According to certain embodiments of the disclosure, two or more instances of computer system 7H00 coupled by communications link 7110 (e.g., LAN, public switched telephone network, or wireless network) may perform the sequence of instructions required to practice embodiments of the disclosure using two or more instances of components of computer system 7H00.

Computer system 7H00 may transmit and receive messages such as data and/or instructions organized into a data structure (e.g., communications packets). The data structure can include program instructions (e.g., application code 7109), communicated through communications link 7110 and communications interface 788. Received program instructions may be executed by data processor 786 as it is received and/or stored in the shown storage device or in or upon any other non-volatile storage for later execution. Computer system in 7H00 may communicate through a data interface 784 to a database server 7103 on an external data repository 7101. Data items in a database may be accessed using a primary key (e.g., a relational database primary key) or the data items may be accessed using simple functional APIs where a key may or may not be used/needed.

Processing element partition 768 is merely one sample partition. Other partitions can include multiple data processors, and/or multiple communications interfaces, and/or multiple storage devices, etc. within a partition. For example, a partition can bound by a multicore processor (e.g., possibly including embedded or collocated memory), or a partition can bound a computing cluster having a plurality of computing elements, any of which computing elements are connected directly or indirectly to a communications link. A first partition can be configured to communicate to a second partition. A particular first partition and particular second partition can be congruent (e.g., in a processing element array) or can be different (e.g., comprising disjoint sets of components).

A module as used herein can be implemented using any mix of any portions of the system memory and any extent of hard-wired circuitry including hard-wired circuitry embodied as a data processor 786. Some embodiments include one or more special-purpose hardware components (e.g., power control, logic, sensors, transducers, etc.). Some embodiments of a module include instructions that are stored in a memory for execution so as to facilitate operational and/or performance characteristics pertaining to the present disclosure. A module may include one or more state machines and/or combinational logic circuits used to implement or facilitate the operational and/or performance characteristics pertaining to the present disclosure.

Various implementations of database server 7103 comprise storage media organized to hold a series of records or files such that individual records or files are accessed using a name or key (e.g., a primary key or a combination of keys and/or query clauses). Such files or records can be organized into one or more data structures (e.g., data structures used to implement or facilitate aspects of the present disclosure. Such files, records, or data structures can be brought into and/or stored in volatile or non-volatile memory. More specifically, the occurrence and organization of the foregoing files, records, and data structures improve the way that the computer stores and retrieves data in memory, for example, to improve the way data is accessed when the computer is performing operations pertaining to the present disclosure, and/or for improving the way data is manipulated when performing computerized operations pertaining to the present disclosure.

FIG. 7I depicts a multi-cloud computing environment 7100. As shown, a first computing cloud (shown as cloud1) is interconnected with a second computing cloud (shown as cloud2). Any known inter-cloud communication technique or combination of inter-cloud communication techniques can be used to facilitate bi-directional data communication that may be demanded when interfacing GAD application simulation and I/O tasks 7118 with inferencing tasks 7119.

In some cases, cloud1 hosts multiple different tenants in a manner such that data corresponding to each of the multiple different tenants may be segregated based on physical or logical boundaries. For example, a first tenant may run a first instance of a GAD application in a first computing platform (e.g., computing platform 718_{T1}), whereas a second tenant may run a second instance of a GAD application in a second computing platform (e.g., computing platform 718_{T2}). In this manner, first tenant-specific data can be securely segregated from any other tenant's tenant-specific data.

In this example partitioning of FIG. 7I, the computing facilities of cloud1 are substantially dedicated to performing GAD application simulation and I/O tasks, whereas the computing facilities of cloud2 are substantially dedicated to performing inferencing. More particularly, the computing facilities of cloud1 perform simulation tasks and then orchestrate communication to/from cloud2 such that the computing facilities of cloud2 can be substantially dedicated to performing inferencing. To do so in this example partitioning, modules situated on cloud1 send training data 7120 to modules of cloud2, and modules of cloud2 send inferencing data 7121 to modules of cloud1.

In the example embodiment as is shown in 7I, the modules of cloud2 are configured particularly so as to be able to perform inferencing tasks using any number of AI supercomputer cores (e.g., AI supercomputer cores 7122₁, AI supercomputer cores 7122₂, AI supercomputer cores, supercomputer cores 7122_{N}), individual ones of, or combinations of the foregoing AI supercomputer cores have core-dedicated local memory. Any individual ones of, or combinations of the AI supercomputer cores can access any of a plurality of decoupled memories (e.g., decoupled memory 7124₁, decoupled memory 7124₂, decoupled memory 7124_{N}). Moreover, individual ones of the AI supercomputer cores can be aggregated into tenant-specific core groups and configured dynamically so as to comport with then-current inferencing and other then-current computing tasks as demanded by particular tenant's operation of that tenant's GAD application. To accommodate such aggregation and dynamic configuration, a core aggregator 7125, possibly in coordination with a corresponding instance of parallelizer 7126 can allocate, aggregate and train a tenant-specific set of the foregoing AI supercomputer cores and/or populate the foregoing decoupled memories with tenant-specific data. Communication of configuration data, training data and inferencing data to and from individual ones of the tenant-specific AI supercomputer cores as well as communication of configuration data, training data and inferencing data to and from individual ones of the tenant-specific set of the foregoing AI supercomputer cores and the decoupled memories can be accomplished by inter-module communications over backplane 7123. Any known techniques for backplane communications in combination with any known technologies for backplane communications can be used. For example, a backplane might be implemented using a high-speed, low-latency fabric based on optical transceivers.

It should be noted that the foregoing AI supercomputer cores can include many millions of perceptrons. Moreover, it should be noted that the foregoing AI supercomputer cores can be configured to accept extremely long training sequences (e.g., tens of thousands of parameters, or hundreds of thousands of parameters, and longer).

FIG. 7J shows a use case 7J00 depicting how one or more human users/experts may interact with components of a multi-platform computing environment. The human interactions with the particular shown computing platforms are being presented merely as examples of how human-generated and/or human-selected information can be used to drive an AI supercomputer. More particularly, the figure is being presented to illustrate how humans may operate respective computing platforms (e.g., computing platform 718_{T3}, and 718_{T9}) so as to prepare data (e.g., AI entity inputs 7140) that is in turn provided to an AI supercomputer.

In this particular use case, the foregoing respective computing platforms are configured to interact with users and/or experts through various user interfaces (e.g., graphical user interfaces, text user interfaces, command line interfaces, etc.) that are purpose-designed to support particular types of interactions. Strictly for purposes of illustration, two types of purpose-specific computing platforms are now briefly described.

Material Specification Platform: A first computing platform 718_{T3} is purpose-designed to support receiving and checking human-generated data (e.g., material specifications). This is depicted by provision of human-determined sets of selected inputs 713 to computing platform 718_{T3}. The human-selected inputs are subjected to (1) semantic checks 7130 as well as (2) completeness checks 7132 over the human-selected inputs. As shown, the foregoing human-determined (e.g., user1-determined) specification of selected inputs 713 may include material requests 704, property requests 706 and possibly priority assignments 7128 that are used in downstream processing, for example to indicated preferences when limitations of the computing equipment or configuration are to be considered. Additionally or alternatively, there may be human-selected provision of experimental learnings 708_{SELECTED}, which selections may include or be influenced by any sorts of measured data such as measurements deriving from human operation of metrology equipment.

In some situations, the foregoing human-determined specification (e.g., values, formats, etc.) of selected inputs 713 may derive, in part, from computerized tools. It is at the choice of the user/expert to decide what specifications or values to provide to computing platform 718_{T3}, and it is at the choice of the user/expert to decide in what form or format to provide. Furthermore, it is at the choice of the user/expert to decide how to select and/or modify certain feedback data 7144 that may be produced by or derived from the results of downstream processing. In some cases feedback data 7144 is of a nature that is most applicable for a user (e.g., user user1, user user2) to consider. In other cases, feedback data 7144 is of a nature that is most applicable for a practitioner to consider.

Prompt Engineering Platform: A second computing platform 718_{T9} is purpose-designed to support human-driven prompt engineering activities. This is depicted by provision of human-determined prompt data 7136 to prompt engineering module 7134 of computing platform 718T9. In addition to processing of human-determined prompt data 7136, prompt engineering module 7134 can further accept human-curated instances of additional prompt data 7136_{ADDITIONAL}, which may derive, in part, from downstream processing (e.g., deriving from the results of processing AI entity inputs tasks 7140 through any number of AI supercomputer cores 7122_{N}).

As is known in the art, configuration of an AI entity, including establishment and biasing of neurons, and including establishment and weighting of inter-neuron connections, can be carried out in a human-supervised manner where a human user/expert specifies the foregoing weights and bias parameters. Alternatively, or in some cases additionally, configuration of an AI entity may be carried out in a semi-supervised manner where a training set may be specified and the foregoing weights and bias parameters are derived from evaluation of the training set. In any case, the AI entity outputs include a human-generated portion 7142 in combination with an AI-generated portion 7138.

Now, returning to the discussion of prompt engineering activities, it is known in the art that even the largest AI supercomputers have structural limitations (e.g., number of parameters) that imply other limitations (e.g., number of tokens in a prompt). Accordingly, a practitioner participates in the overall flow in a manner that facilitates generation of a prompt that is both effective in providing a meaningful and effective prompt to the AI entity while at the same time comporting to AI supercomputer limitations. To this end, revised material requests 704_{REVISED} may be considered, and/or revised property requests 706_{REVISED} may be considered when forming a meaningful and effective prompt to the AI entity.

Based on the foregoing, it is to be understood that a user enables an AI system through user input. As has been discussed, user may contribute their input in the form of dictating material classes and properties (such as in the initial dataset). In one embodiment, the Ai system may use intent recognition in identifying the user's goals and/or inputs, which in turn may guide subsequent actions. Further, the inputs provided may additionally include contextual understanding for the AI system to ensure that the AI system appreciates the ongoing analysis (such as referencing prior inputs and acknowledging user preferences). In cases where additional information is required, the AI system may retrieve data from various sources and machine learning models (which, again, may be trained on user dictated material classes and properties).

In one embodiment, it is to be appreciated that the core of the AI's functionality may reside in processing and decision-making, which again, is based initially on user dictated input. Further, many of the outputs from the AI system may be presented to the user for continued input through the process. In this manner, the overall system disclosed herein may benefit from both user dictated input and AI processing power, where the AI system may otherwise function as a digital laboratory of testing atomic structures.

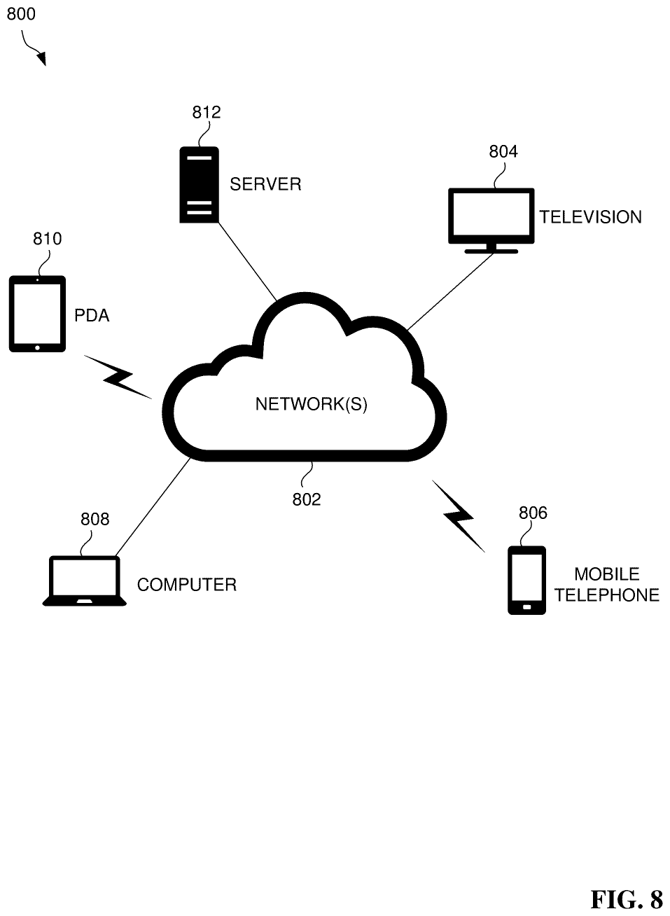

FIG. 8 illustrates a network architecture 800, in accordance with one possible embodiment. As shown, at least one network 802 is provided. In the context of the present network architecture 800, the network 802 may take any form including, but not limited to a telecommunications network, a local area network (LAN), a wireless network, a wide area network (WAN) such as the Internet, peer-to-peer network, cable network, etc. While only one network is shown, it should be understood that two or more similar or different networks 802 may be provided.

Coupled to the network 802 is a plurality of devices. For example, a server computer 812 and an end user computer 808 may be coupled to the network 802 for communication purposes. Such end user computer 808 may include a desktop computer, lap-top computer, and/or any other type of logic. Still yet, various other devices may be coupled to the network 802 including a personal digital assistant (PDA) device 810, a mobile phone device 806, a television 804, etc.

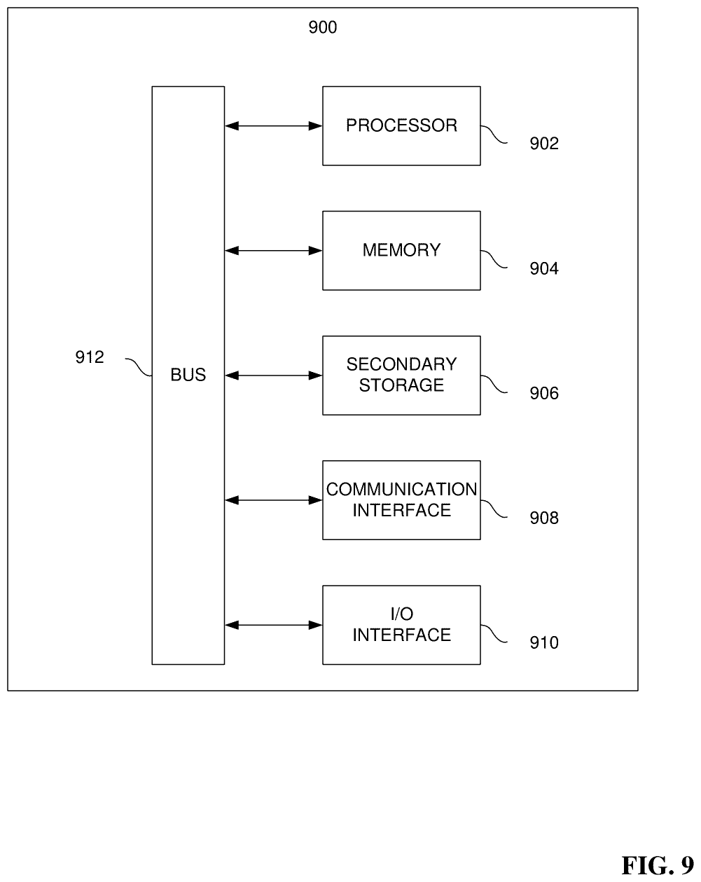

FIG. 9 illustrates an exemplary system 900, in accordance with one embodiment. As an option, the system 900 may be implemented in the context of any of the devices of the network architecture 800 of FIG. 8. Of course, the system 900 may be implemented in any desired environment.

As shown, a system 900 is provided including at least one central processor 902 which is connected to a communication bus 912. The system 900 also includes main memory 904 [e.g., random access memory (RAM), etc.]. The system 900 also includes a graphics processor 908 and a display 910.

The system 900 may also include a secondary storage 906. The secondary storage 906 includes, for example, a hard disk drive and/or a removable storage drive, representing a floppy disk drive, a magnetic tape drive, a compact disk drive, etc. The removable storage drive reads from and/or writes to a removable storage unit in a well known manner.

Computer programs, or computer control logic algorithms, may be stored in the main memory 904, the secondary storage 906, and/or any other memory, for that matter. Such computer programs, when executed, enable the system 900 to perform various functions (as set forth above, for example). Memory 904, storage 906 and/or any other storage are possible examples of non-transitory computer-readable media. It is noted that the techniques described herein, in an aspect, are embodied in executable instructions stored in a computer readable medium for use by or in connection with an instruction execution machine, apparatus, or device, such as a computer-based or processor-containing machine, apparatus, or device. It will be appreciated by those skilled in the art that for some embodiments, other types of computer readable media are included which may store data that is accessible by a computer, such as magnetic cassettes, flash memory cards, digital video disks, Bernoulli cartridges, random access memory (RAM), read-only memory (ROM), and the like.

As used here, a “computer-readable medium” includes one or more of any suitable media for storing the executable instructions of a computer program such that the instruction execution machine, system, apparatus, or device may read (or fetch) the instructions from the computer readable medium and execute the instructions for carrying out the described methods. Suitable storage formats include one or more of an electronic, magnetic, optical, and electromagnetic format. A non-exhaustive list of conventional exemplary computer readable medium includes: a portable computer diskette; a RAM; a ROM; an erasable programmable read only memory (EPROM or flash memory); optical storage devices, including a portable compact disc (CD), a portable digital video disc (DVD), a high definition DVD (HD-DVD™), a BLU-RAY disc; and the like.

It should be understood that the arrangement of components illustrated in the Figures described are exemplary and that other arrangements are possible. It should also be understood that the various system components (and means) defined by the claims, described below, and illustrated in the various block diagrams represent logical components in some systems configured according to the subject matter disclosed herein.

For example, one or more of these system components (and means) may be realized, in whole or in part, by at least some of the components illustrated in the arrangements illustrated in the described Figures. In addition, while at least one of these components are implemented at least partially as an electronic hardware component, and therefore constitutes a machine, the other components may be implemented in software that when included in an execution environment constitutes a machine, hardware, or a combination of software and hardware.

More particularly, at least one component defined by the claims is implemented at least partially as an electronic hardware component, such as an instruction execution machine (e.g., a processor-based or processor-containing machine) and/or as specialized circuits or circuitry (e.g., discreet logic gates interconnected to perform a specialized function). Other components may be implemented in software, hardware, or a combination of software and hardware. Moreover, some or all of these other components may be combined, some may be omitted altogether, and additional components may be added while still achieving the functionality described herein. Thus, the subject matter described herein may be embodied in many different variations, and all such variations are contemplated to be within the scope of what is claimed.

In the description above, the subject matter is described with reference to acts and symbolic representations of operations that are performed by one or more devices, unless indicated otherwise. As such, it will be understood that such acts and operations, which are at times referred to as being computer-executed, include the manipulation by the processor of data in a structured form. This manipulation transforms the data or maintains it at locations in the memory system of the computer, which reconfigures or otherwise alters the operation of the device in a manner well understood by those skilled in the art. The data is maintained at physical locations of the memory as data structures that have particular properties defined by the format of the data. However, while the subject matter is being described in the foregoing context, it is not meant to be limiting as those of skill in the art will appreciate that various of the acts and operations described hereinafter may also be implemented in hardware.

To facilitate an understanding of the subject matter described herein, many aspects are described in terms of sequences of actions. At least one of these aspects defined by the claims is performed by an electronic hardware component. For example, it will be recognized that the various actions may be performed by specialized circuits or circuitry, by program instructions being executed by one or more processors, or by a combination of both. The description herein of any sequence of actions is not intended to imply that the specific order described for performing that sequence must be followed. All methods described herein may be performed in any suitable order unless otherwise indicated herein or otherwise clearly contradicted by context.

The use of the terms “a” and “an” and “the” and similar referents in the context of describing the subject matter (particularly in the context of the following claims) are to be construed to cover both the singular and the plural, unless otherwise indicated herein or clearly contradicted by context. Recitation of ranges of values herein are merely intended to serve as a shorthand method of referring individually to each separate value falling within the range, unless otherwise indicated herein, and each separate value is incorporated into the specification as if it were individually recited herein. Furthermore, the foregoing description is for the purpose of illustration only, and not for the purpose of limitation, as the scope of protection sought is defined by the claims as set forth hereinafter together with any equivalents thereof entitled to. The use of any and all examples, or exemplary language (e.g., “such as”) provided herein, is intended merely to better illustrate the subject matter and does not pose a limitation on the scope of the subject matter unless otherwise claimed. The use of the term “based on” and other like phrases indicating a condition for bringing about a result, both in the claims and in the written description, is not intended to foreclose any other conditions that bring about that result. No language in the specification should be construed as indicating any non-claimed element as essential to the practice of the invention as claimed.

The embodiments described herein included the one or more modes known to the inventor for carrying out the claimed subject matter. Of course, variations of those embodiments will become apparent to those of ordinary skill in the art upon reading the foregoing description. The inventor expects skilled artisans to employ such variations as appropriate, and the inventor intends for the claimed subject matter to be practiced otherwise than as specifically described herein. Accordingly, this claimed subject matter includes all modifications and equivalents of the subject matter recited in the claims appended hereto as permitted by applicable law. Moreover, any combination of the above-described elements in all possible variations thereof is encompassed unless otherwise indicated herein or otherwise clearly contradicted by context.

What is claimed is:

1. A method comprising:
receiving, at a machine learning system, two or more molecular structures from at least one structural dataset, wherein the two or more molecular structures relate to ionic mobility;
calculating, using the machine learning system, atomic weights for the two or more molecular structures;
training the machine learning system based on the two or more molecular structures, wherein the training relies on at least one intrinsic atomic feature and the calculated atomic weights for the two or more molecular structures;
applying, using the machine learning system, a bias-correction for the two or more molecular structures; and
outputting, using the machine learning system, one or more molecular dynamics (MD) simulations for the two or more molecular structures, wherein the one or more MD simulations including validating associated structural energy or atomic forces for the two or more molecular structures.

2. The method of claim 1, wherein the two or more molecular structures include at least one of: atomic positions, associated structural energy, or atomic forces affecting one or more atomic structures of the two or more molecular structures.

3. The method of claim 1, wherein the receiving includes initial data obtained using ab-initio simulation via a density functional theory (DFT) calculation.

4. The method of claim 1, wherein the training includes converting the two or more molecular structures into one or more mathematical descriptors.

5. The method of claim 4, wherein the converting includes using translationally invariant functions.

6. The method of claim 4, wherein the converting includes using rotationally invariant functions.

7. The method of claim 1, wherein the training includes using a machine learned interatomic potentials (MLIP) model using a traditional ensemble.

8. The method of claim 1, wherein the training includes using a machine learned interatomic potentials (MLIP) model using a diverse ensemble.

9. The method of claim 1, wherein the at least one factor includes at least one of: atomic positions, associated structural energy, or atomic forces affecting the two or more molecular structures.

10. The method of claim 1, wherein the outputting includes evaluating data from the training with an ensemble of networks to evaluate uncertainty levels against known threshold values.

11. The method of claim 10, wherein the threshold is a known eV/angstrom value for atomic forces.

12. The method of claim 10, wherein the threshold is a known meV/angstrom value for structural energy.

13. The method of claim 10, wherein the evaluating of uncertainty levels against known threshold values indicates a level of uncertainty in ionic conductivity of the ionic mobility.

14. The method of claim 1, further comprising adding one or more new structural datasets to a first database.

15. The method of claim 14, further comprising adding the one or more new structural datasets to a second database that is different from the first database.

16. The method of claim 15, wherein the one or more new structural datasets in the second database are re-evaluated using a DFT process and are outputted as additional molecular structures in the at least one structural dataset.

17. The method of claim 2, wherein the MD simulations for the two or more molecular structures includes increasing a temperature of the one or more MD simulations.

18. The method of claim 2, wherein the MD simulations for the two or more molecular structures includes decreasing a temperature of the one or more MD simulations.

19. The method of claim 2, wherein the MD simulations for the two or more molecular structures includes determining an activation energy based on increasing or decreasing a temperature of the one or more atomic structures.

20. The method of claim 1, wherein at least one of the validated associated structural energy or the validated atomic forces is used to improve a model associated with the machine learning system, wherein the model is machine learned interatomic potentials (MLIP).

21. A system, comprising:
a non-transitory memory storing instructions; and
one or more processors in communication with the non-transitory memory, wherein the one or more processors execute the instructions to:
receive, at a machine learning system, two or more molecular structures from at least one structural dataset, wherein the two or more molecular structures relate to ionic mobility;
calculate, using the machine learning system, atomic weights for the two or more molecular structures;
train the machine learning system based on the two or more molecular structures, wherein the training relies on at least one intrinsic atomic feature and the calculated atomic weights for the two or more molecular structures;
apply, using the machine learning system, a bias-correction for the two or more molecular structures; and
output, using the machine learning system, one or more molecular dynamics (MD) simulations for the two or more molecular structures, wherein the one or more MD simulations including validating associated structural energy or atomic forces for the two or more molecular structures.

22. A computer program product comprising computer executable instructions stored on a non-transitory computer readable medium that when executed by a processor instruct the processor to:
receive, at a machine learning system, two or more molecular structures from at least one structural dataset, wherein the two or more molecular structures relate to ionic mobility;
calculate, using the machine learning system, atomic weights for the two or more molecular structures;
train the machine learning system based on the two or more molecular structures, wherein the training relies on at least one intrinsic atomic feature and the calculated atomic weights for the two or more molecular structures;
apply, using the machine learning system, a bias-correction for the two or more molecular structures; and
output, using the machine learning system, one or more molecular dynamics (MD) simulations for the two or more molecular structures, wherein the one or more MD simulations including validating associated structural energy or atomic forces for the two or more molecular structures.
